(19) United States
(12) Patent Application Publication
(10) Pub. No.: US 2025/0093329 A1
(43) Pub. Date: Mar. 20, 2025

(54) SCREENING OF COMPOUNDS

(71) Applicant: THE UNIVERSITY OF TULSA, Tulsa, OK (US)

(72) Inventors: Robert J. Sheaff, Owasso, OK (US); Angus A. Lamar, Tulsa, OK (US); Ian Mitchell, Bartlesville, OK (US)

(21) Appl. No.: 18/832,761

(22) PCT Filed: Jan. 25, 2023
(86) PCT No.: PCT/US2023/011528

§ 371 Date: Jul. 24, 2024

Related U.S. Application Data
(60) Provisional application No. 63/303,350, filed on Jan. 26, 2022.

(51) Int. Cl. G01N 33/50

(52) U.S. Cl. CPC G01N 33/5011

(57) ABSTRACT

Disclosed is a method for preparing patient derived cancer cells in a patient derived serum and/or plasma and individualized cancer cell support media containing amino acids, vitamins, inorganic salts and glucose in concentrations corresponding to the organ in which the cancer appears. Also disclosed is a method for screening compounds to determine the effectiveness of those compounds in treating the patient's specific cancer.

### BACKGROUND

While advancements have been made in the treatment of various cancers, most treatments have been developed for wide application to all patients. A targeted approach, wherein anti-cancer compounds or any FDA approved drugs are selected for an individual's unique cancer and unique body chemistry remains a challenge. To achieve this goal will require the ability to use cancer cells from the patient in conjunction with new screening methods to identify those compounds capable of killing the cancer without damaging the patient's healthy, normal cells. Further, the ability to transport the patient's cells to the testing lab must be improved to ensure cell viability for a period of time to carry out the necessary tests. Finally, the screening methods need to occur in a rapid manner to permit treatment of the patient as soon as possible.

### SUMMARY

Disclosed is a method for assessing the in vitro effectiveness of a compound, or a mixture of compounds, against a specific cancer type. The method includes the steps of:
obtaining cancer cells from a patient;
preparing individualized cancer cell support media formulated to correspond to an organ within the patient from which the cancer cells were obtained;
maintaining the cancer cells in the individualized cancer cell support media under conditions which will promote cancer cell survival and growth using metabolic pathways corresponding to cancer cell growth in the patient;
treating the cancer cells with a compound or compounds of interest;
adding a luminescing agent to the cancer cells;
measuring a level of light emission of the luminescing agent and comparing the level of light emission to a level of light emission of a control;
determining an IC₅₀ value for the compound or compounds of interest thereby assessing the in vitro effectiveness of a compound against a specific cancer type.

Also disclosed is a method for preparing patient derived cancer cells. The method includes the steps of:
obtaining cancer cells from a patient;
preparing individualized cancer cell support media containing, patient derived serum and/or plasma, amino acids, vitamins, inorganic salts, glucose in concentrations which correspond to an organ within the patient from which the cancer cells were obtained;
incubating the cancer cells in the individualized cancer cell support media at temperatures between 35° C. and 39° C. under an atmosphere containing carbon dioxide for a period of 12 hours to 18 hours thereby promoting cancer cell growth using metabolic pathways corresponding to cancer cell growth in the patient.

Further disclosed is a method for controlling the metabolic pathways used by cells during in vitro testing. The method includes the steps of:
identifying a cancer cell type to be treated;
culturing cells of the identified cancer cell type in a patient specific cell support media formulated, the patient specific support media corresponds to the in vivo environment of an organ within the patient that corresponds to the cancer cell type;
screening at least one compound of interest to ensure that the compound does not inhibit a luciferase enzyme;
upon confirmation that the at least one compound of interest does not inhibit the luciferase enzyme, treating the cells of the identified cancer cell type with the at least one compound of interest;
after treating the cells of the identified cancer cell type, monitoring the ATP level of the cells of the identified cancer cell type to determine cytotoxicity of the at least one compound of interest towards the cells of the identified cancer cell type.

### BRIEF DESCRIPTION OF THE DRAWINGS

FIGS. 1A-1L provide the chemical structures of the N-benzyl sulfonamides identified in FIG. 4 as well as other N-benzyl sulfonamides.

FIG. 2 provides the initial cell viability test results for the screening of compounds 2, 5-9, 11-12, 14, 16, 18-22 of FIGS. 1A and 1B where each compound was tested at concentrations of 500 μM and 100 μM.

FIG. 3 provides the results of the cytotoxicity screening of compounds 2, 5-9, 11-12, 14, 16, 18-22 of FIGS. 1A and 1B performed according to the prior art method for determining cytotoxicity.

FIG. 4 provides the IC₅₀ results for the indicated compounds from FIGS. 7A-3 prepared according to the disclosed method for determining ATP levels following treatment with two component compositions compared to two known anti-cancer compounds ABT-751 and Indisulam.

FIG. 5 provides the structure of rotenone.

FIG. 6 provides the structure of 2-deoxyglucose.

FIG. 7 depicts the light emitting reaction of D-Luciferin in the presence of Firefly Luciferase.

FIG. 8 depicts the structure and components of N-benzyl sulfonamide.

FIG. 9 provides four non-limiting examples of N-benzyl sulfonamides where the R³ group is an indole and the sulfonamide component is attached at different locations on the indole.

FIG. 10 provides non-limiting examples of the N-substrate.

FIG. 11 provides the chemical structures of compounds tested according to the disclosed method.

FIG. 12 provides the chemical structures of compounds tested according to the disclosed method.

FIG. 13 is a graph depicting the measurement of ATP using CellTiter-Glo using a variable number of cells.

FIG. 14 demonstrates the depletion of ATP levels over time for cells under varying conditions.

FIG. 15 provides a comparison of traditional cytotoxicity testing the improved testing method described herein using HEK293 cells (kidney cancer), DMEM media, and the library sulfonamide compounds 1-30 found in FIGS. 1A-1C.

FIGS. 16-33 provide test data for testing of compounds 140-161 found in FIGS. 11 and 12 against various cell lines under traditional testing and the rapid testing method disclosed herein.

### DETAILED DESCRIPTION

Throughout this disclosure, the terms “about”, “approximate”, and variations thereof, are used to indicate that a value includes the inherent variation or error for the device, system, the method being employed to determine the value, or the variation that exists among the study subjects.

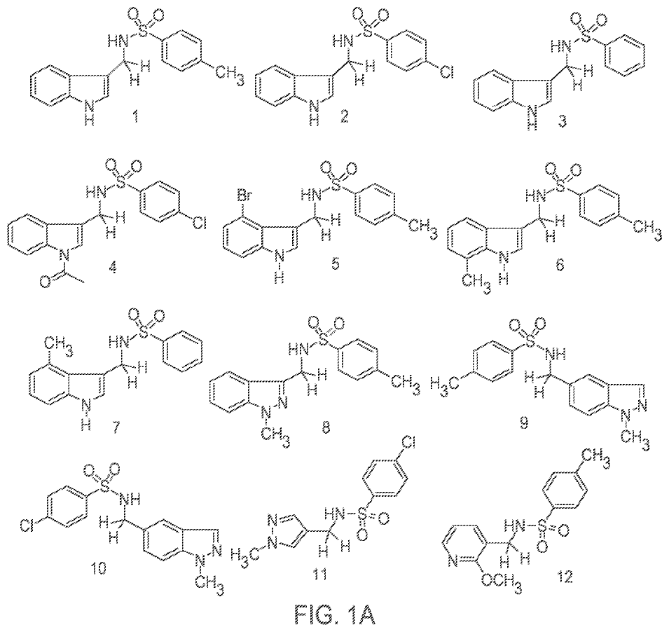

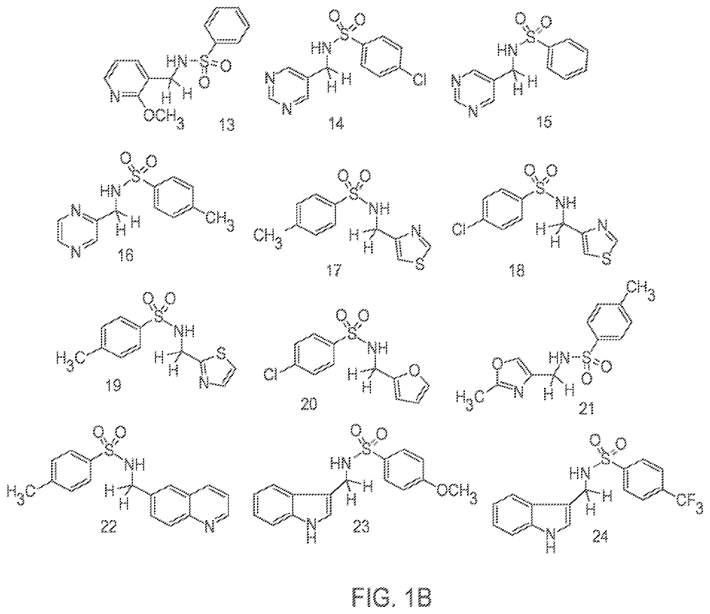

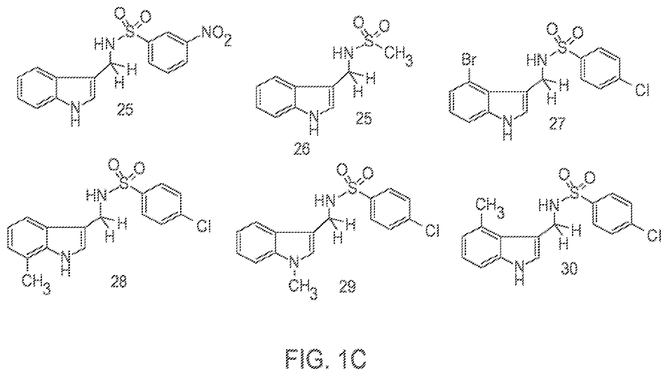

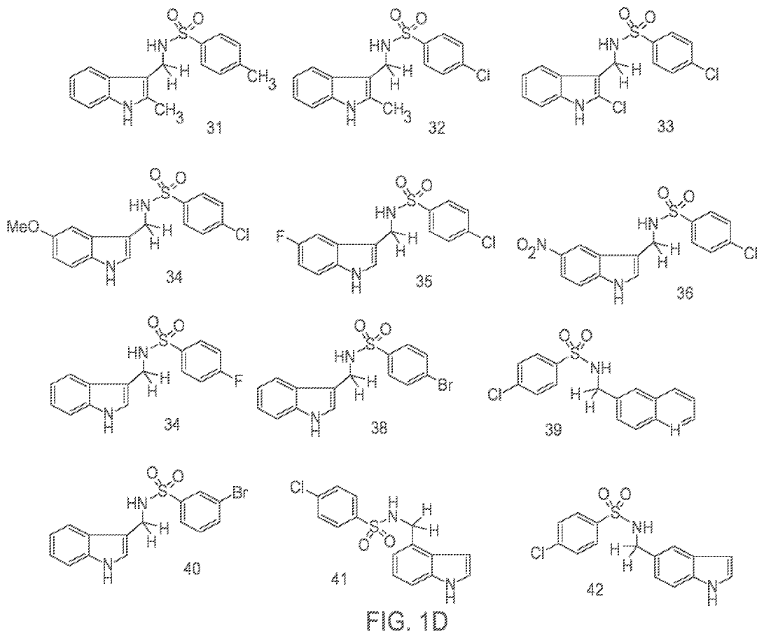

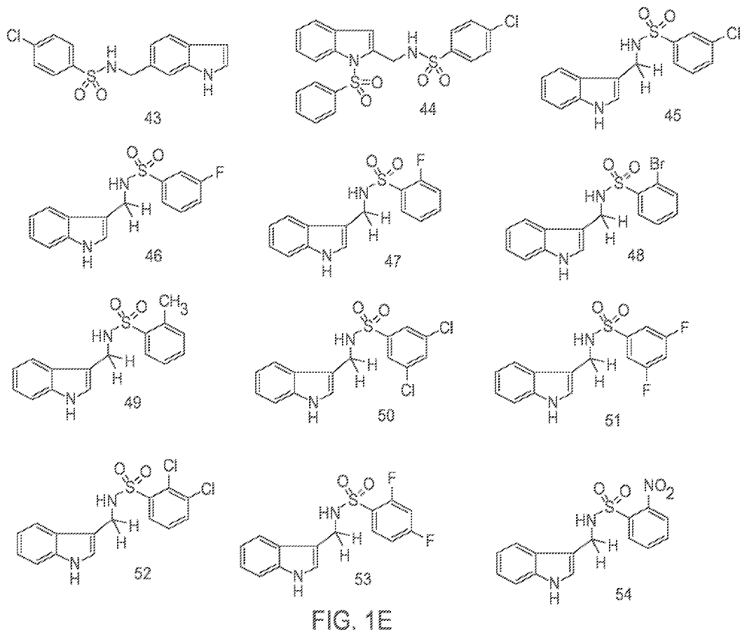

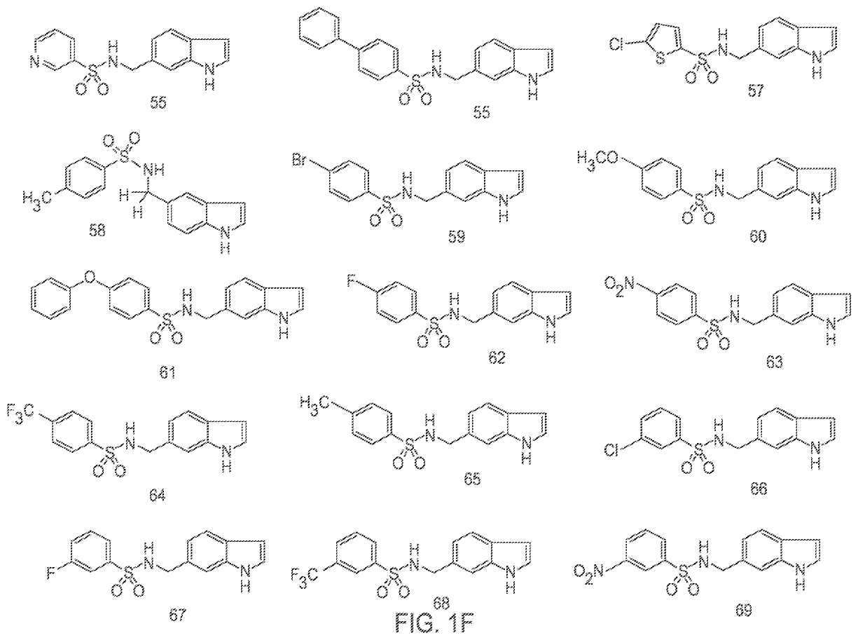

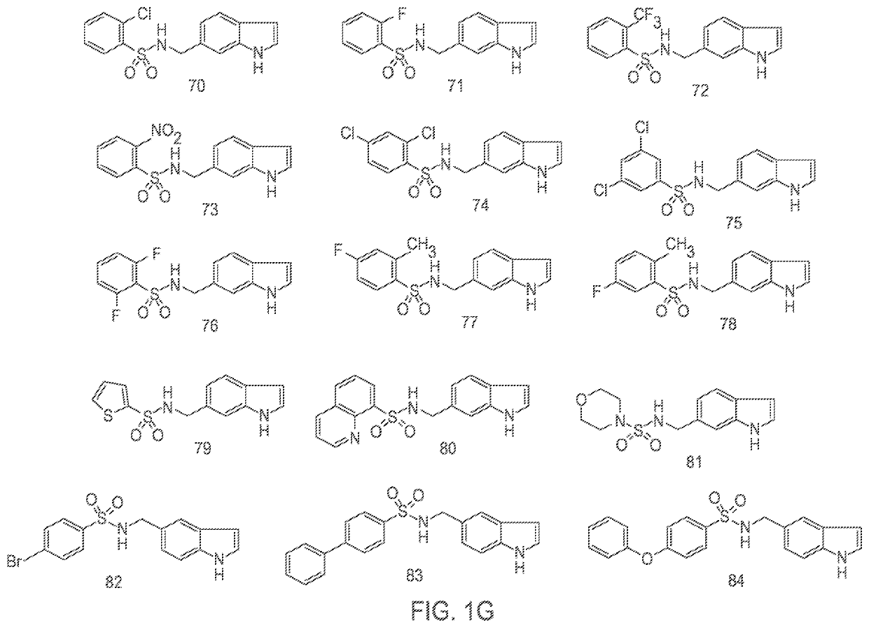

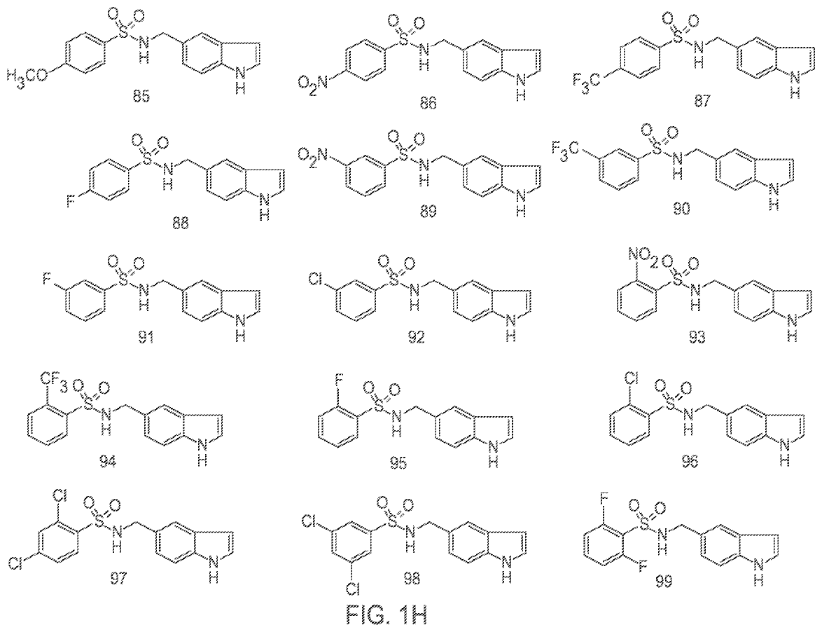

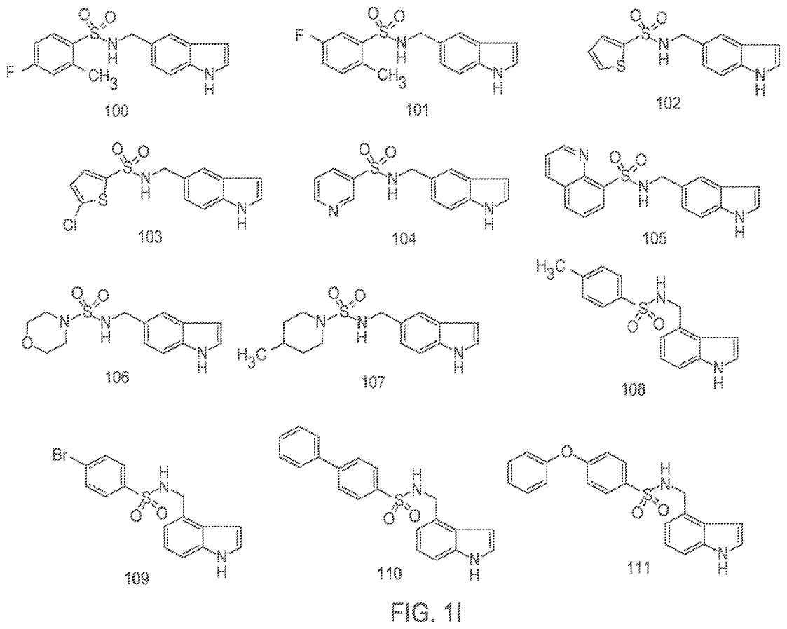

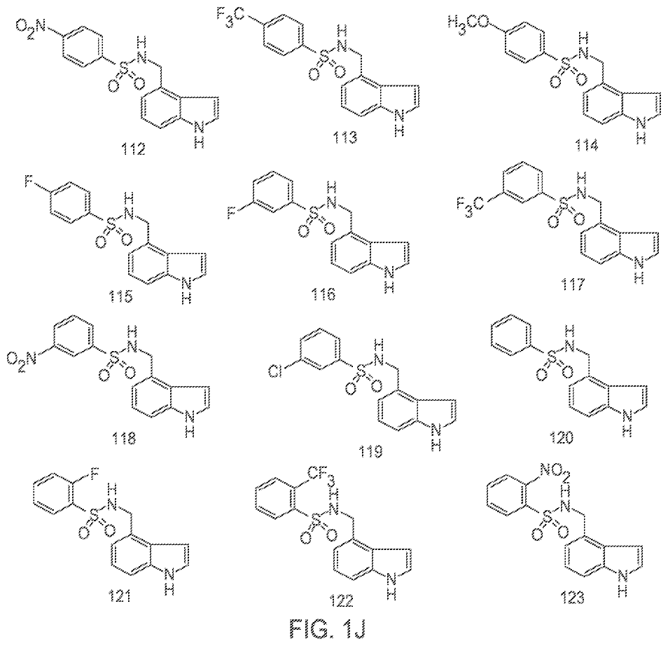

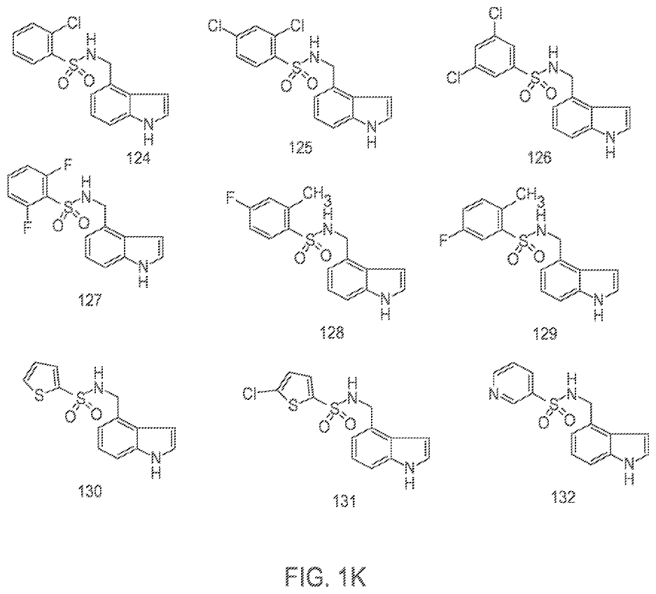

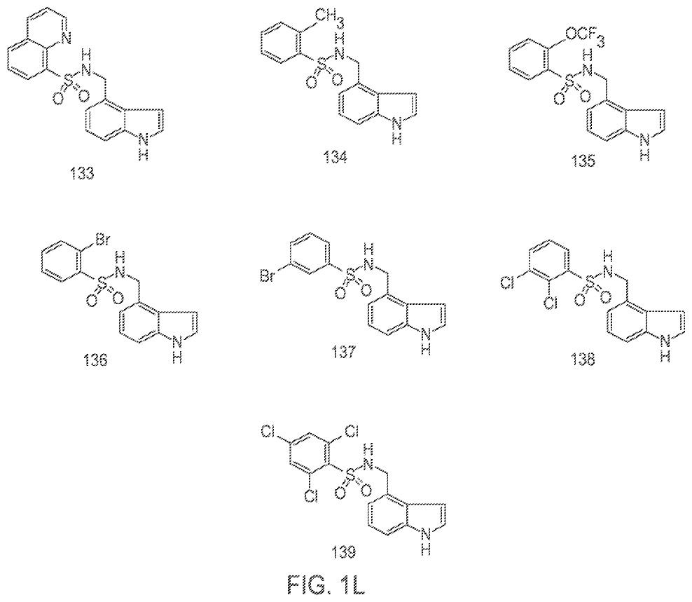

### Example Compounds 1-139 as Depicted in FIGS. 1A-L

The following examples demonstrate the use of a variety of heteroaromatic compounds as the R³ scaffold material for production of N-benzyl-sulfonamides and a variety of N-substrates with functional groups R¹. The final products are depicted as compounds 1-30 in FIGS. 1A-L. The number in parentheses appearing after the title of the compound identifies the corresponding structure in FIGS. 1A-1L.
N-[(1H-indol-3-yl)methyl]-4-methylbenzenesulfonamide (1). Brown-red solid (24 mg, 61%): m.p. 140-144° C.; purification (hexanes:EtOAc, 60:40), R_{f}=0.42. ¹H NMR (400 MHz, CDCl₃): δ=8.06 (bs, 1H), 7.79 (dt, J=8.2 Hz, J=2.0 (×2) Hz, 2H), 7.40 (dd, J=8.0, 1.0 Hz, 1H), 7.35-7.29 (m, 3H), 7.20 (ddd, J=8.2, J=7.0, J=1.2 Hz, 1H), 7.08 (ddd, J=8.2, J=7.0, J=1.2 Hz, 1H), 7.05 (d, J=2.3 Hz, 1H), 4.47 (bt, J=5.5 Hz, 1H), 4.32 (d, J=5.5 Hz, 2H), 2.45 (s, 3H) ppm. ¹³C NMR (100 MHz, CDCl₃): δ=143.4, 136.7, 136.2, 129.7, 127.3, 126.1, 123.3, 122.7, 120.0, 118.6, 111.3, 111.0, 39.0, 21.6 ppm. IR (neat): v=3377, 3275, 3059, 2921, 1724, 1596, 1287, 1153, 1021, 744 cm⁻¹. HRMS (ESI): calculated for C₁₆H₁₇N₂O₂S₁ [M+H]⁺ requires m/z 301.10108, found m/z 301.06378.
N-[(1H-indol-3-yl)methyl]-4-chlorobenzenesulfonamide (2). Brown-red solid (48 mg, 60%): m.p. 154-158° C.; purification (hexanes:EtOAc, 70:30), R_{f}=0.22. ¹H NMR (400 MHz, CDCl₃): δ=8.05 (bs, 1H), 7.79 (dt, J=8.6 Hz, J=2.0 (×2) Hz, 2H), 7.47-7.39 (m, 3H), 7.34 (d, J=8.2 Hz, 1H), 7.21 (ddd, J=8.2, J=7.4, J=1.2 Hz, 1H), 7.10 (ddd, J=7.8, J=7.0, J=0.8 Hz, 1H), 7.05 (d, J=2.3 Hz, 1H), 4.56 (bt, J=5.5 Hz, 1H), 4.36 (d, J=5.5 Hz, 2H) ppm. ¹³C NMR (100 MHz, CDCl₃): δ=139.0, 138.4, 136.2, 129.2, 128.6, 126.0, 123.4, 122.8, 120.2, 118.5, 111.4, 110.7, 39.1 ppm. IR (neat): v=3406, 3281, 3093, 2918, 2849, 1699, 1418, 1317, 1157, 1014, 824, 742 cm⁻¹. HRMS (ESI): calculated for C₁₅H₁₄N₂O₂S₁Cl₁[M+H]⁺ requires m/z 321.04645, found m/z 321.04547.
N-[(1H-indol-3-yl)methyl]benzenesulfonamide (3). Tan solid (22 mg, 60%): m.p. 132-140° C.; purification (hexanes:EtOAc, 60:40), R_{f}=0.33. ¹H NMR (400 MHz, CDCl₃): δ=8.05 (bs, 1H), 7.93-7.89 (m, 2H), 7.62-7.57 (m, 1H), 7.55-7.49 (m, 2H), 7.39 (d, J=8.2 Hz, 1H), 7.34 (d, J=8.2 Hz, 1H), 7.20 (ddd, J=8.2 Hz, J=7.4 Hz, J=1.2 Hz, 1H), 7.08 (ddd, J=8.2 Hz, J=7.0 Hz, J=1.2 Hz, 1H), 7.03 (d, J=2.3 Hz, 1H), 4.51 (bt, J=5.1 Hz, 1H), 4.35 (d, J=5.1 Hz, 2H) ppm. ¹³C NMR (100 MHz, CDCl₃): δ=139.8, 136.2, 132.6, 129.1, 127.2, 126.1, 123.3, 122.7, 120.1 118.6, 111.3, 110.9, 39.0 ppm. IR (neat): v=3394, 3242, 2918, 1700, 1447, 1423, 1333, 1224, 1153, 730 cm⁻¹. HRMS (ESI): calculated for C₁₅H₁₅N₂O₂S₁ [M+H]⁺ requires m/z 287.08543, found m/z 287.08429.
N-[(1-acetyl-1H-indole-3-yl)methyl]-4-chlorobenzenesulfonamide (4). Off-white solid (30 mg, 64%): m.p. 176-180° C.; purification (hexanes:EtOAc, 60:40), R_{f}=0.29. ¹H NMR (400 MHz, CDCl₃): δ=8.36 (bd, J=8.2 Hz, 1H), 7.78 (dt, J=8.6 Hz, J=2.0 (×2) Hz, 2H), 7.44 (dt, J=8.6 Hz, J=2.0 (×2) Hz, 2H), 7.41 (d, J=7.4 Hz, 1H), 7.36 (ddd, J=8.3 Hz, J=7.1 Hz, J=1.4 Hz, 1H), 7.28-7.23 (m, 2H), 4.80 (bt, J=5.5 Hz, 1H), 4.31 (d, J=5.5 Hz, 2H), 2.55 (s, 3H) ppm. ¹³C NMR (100 MHz, CDCl₃): δ=168.3, 139.4, 138.2, 135.9, 129.4, 128.6, 128.5, 125.9, 123.9, 118.7, 117.2, 116.8, 38.8, 23.9 ppm. IR (neat): v=3222, 3112, 2924, 1676, 1451, 1158, 752 cm⁻¹. HRMS (ESI): calculated for C₁₇H₁₆N₂O₃S₁Cl₁ [M+H]⁺ requires m/z 363.05702, found m/z 363.05640.
N-[(4-bromo-1H-indol-3-yl)methyl]-4-methylbenzenesulfonamide (5). Brown solid (16 mg, 34%): m.p. 117-122° C.; purification (hexanes:EtOAc, 60:40), R_{f}=0.22. ¹H NMR (400 MHz, CDCl₃): δ=8.21 (bs, 1H), 7.64 (d, J=8.2 Hz, 2H), 7.23 (dd, J=8.2 Hz, J=0.8 Hz, 1H), 7.20 (dd, J 8.2 Hz, J=0.8 Hz, 1H), 7.15-7.12 (m, 3H), 6.98 (t, J=7.8 Hz, 1H), 4.99 (bt, J=5.9 Hz, 1H), 4.49 (d, J=5.9 Hz, 2H), 2.34 (s, 3H) ppm. ¹³C NMR (100 MHz, CDCl₃): δ=142.9, 137.5, 137.2, 129.2, 126.9, 126.0, 124.9, 124.0, 123.2, 113.1, 111.5, 110.8, 39.3, 21.4 ppm. IR (neat): v=3326, 2916, 2848, 1639, 1511, 1387, 1294, 1151, 731 cm⁻¹. HRMS (ESI): calculated for C₁₆H₁₆N₂O₂S₁Br₁ [M+H]⁺ requires m/z 379.01159, found m/z 379.01059.
N-[(7-methyl-1H-indol-3-yl)methyl]benzenesulfonamide (6). Off-white solid (23 mg, 58%): m.p. 176-179° C.; purification (hexanes: EtOAc, 60:40), R_{f}=0.38. ¹H NMR (400 MHz, CDCl₃): δ=8.02 (bs, 1H), 7.77 (d, J=8.2 Hz, 2H), 7.30 (d, J=8.2 Hz, 2H), 7.23 (t, J=4.5 Hz, 1H), 7.04 (d, J=2.3 Hz, 1H), 7.00 (d, J=5.5 Hz, 2H), 4.55 (bt, J=5.5 Hz, 1H), 4.30 (d, J=5.5 Hz, 2H), 2.45 (s, 3H), 2.44 (s, 3H) ppm. ¹³C NMR (100 MHz, CDCl₃): δ=143.4, 136.7, 135.8, 129.7, 127.3, 126.5, 125.7, 123.1, 120.5, 120.2, 116.3, 111.4, 39.1, 21.5, 16.6 ppm. IR (neat): v=3386, 3269, 3053, 2917, 1597, 1412, 1300, 1153, 1091, 1041, 909, 810, 736, 668, 540 cm⁻¹. HRMS (ESI): calculated for C₁₇H₁₉N₂O₂S₁ [M+H]⁺ requires m/z 315.11673, found m/z 315.10825.
N-[(4-methyl-1H-indol-3-yl)methyl]benzenesulfonamide (7). Brown solid (24 mg, 63%): m.p. 130-136° C.; purification (hexanes:EtOAc, 60:40), R_{f}=0.34. ¹H NMR (400 MHz, CDCl₃): δ=8.06 (bs, 1H), 7.92-7.88 (m, 2H), 7.62-7.56 (m, 1H), 7.55-7.48 (m, 2H), 7.16 (d, J=7.8 Hz, 1H), 7.07 (t, J=7.4 Hz, 1H), 6.98 (d, J=2.7 Hz, 1H), 6.86-6.82 (m, 1H), 4.51 (m, 1H), 4.41 (d, J=5.1 Hz, 2H), 2.54 (s, 3H) ppm. ¹³C NMR (100 MHz, CDCl₃): δ=141.9, 139.6, 136.7, 132.6, 130.4, 129.1, 127.2, 126.4, 124.9, 122.6, 121.5, 111.1, 109.3, 40.4, 19.7 ppm. IR (neat): v=3271, 1701, 1446, 1413, 1310, 1154, 1090, 1028, 747, 686 cm⁻¹. HRMS (ESI): calculated for C₁₆H₁₇N₂O₂S₁[M+H]⁺ requires m/z 301.10108, found m/z 301.09982.
4-methyl-N-[(1-methyl-1H-indazol-3-yl)methyl]benzenesulfonamide (8). White solid (26 mg, 66%): m.p. 129-131° C.; purification (hexanes:EtOAc, 60:40), R_{f}=0.26. ¹H NMR (400 MHz, CDCl₃): δ=7.74 (dt, J=8.2 Hz, J=2.0 (×2) Hz, 2H), 7.66 (dt, J=8.2 Hz, J=1.0 Hz, 1H), 7.38 (ddd, J=8.2 Hz, J=6.7 Hz, J=0.8 Hz, 1H), 7.29 (dt, J=8.2 Hz, J=0.8 Hz, 1H), 7.25-7.22 (m, 2H), 7.12 (ddd, J=8.0 Hz, J=6.7 Hz, J=0.8 Hz, 1H), 5.14 (bt, J=5.9 Hz, 1H), 4.47 (d, J=5.9 Hz, 2H), 3.93 (s, 3H), 2.39 (s, 3H) ppm. ¹³C NMR (100 MHz, CDCl₃): δ=143.4, 140.9, 139.2, 136.5, 129.6, 127.2, 126.7, 121.7, 120.6, 120.2, 109.0, 40.1, 35.3, 21.5 ppm IR (neat): v=3087, 2868, 1599, 1441, 1322, 1153, 1078, 1048, 800, 656 cm⁻¹. HRMS (ESI): calculated for C₁₆H₁₈N₃O₂S₁ [M+H]⁺ requires m/z 316.11197, found m/z 316.11053.
4-methyl-N-[(1-methyl-1H-indazol-5-yl)methyl]benzenesulfonamide (9). White solid (26 mg, 60%): m.p. 147-150° C.; purification (hexanes:EtOAc, 60:40), R_{f}=0.21. ¹H NMR (400 MHz, CDCl₃): δ=7.88 (s, 1H), 7.77 (dt, J=8.2 Hz, J=2.0 (×2) Hz, 2H), 7.50 (s, 1H), 7.33-7.24 (m, 4H), 4.74 (bt, J=5.9 Hz, 1H), 4.22 (d, J=5.9 Hz, 2H), 4.05 (s, 3H), 2.43 (s, 3H) ppm. ¹³C NMR (100 MHz, CDCl₃): δ=143.5, 139.5, 136.9, 132.6, 129.7, 128.4, 127.2, 126.7, 123.9, 120.4, 109.4, 47.5, 35.6, 21.5 ppm. IR (neat): v=3166, 2931, 1512, 1327, 1156, 1055, 800, 750, 660 cm⁻¹. HRMS (ESI): calculated for C₁₆H₁₈N₃O₂S₁ [M+H]⁺ requires m/z 316.11197, found m/z 316.11096.
4-chloro-N-[(1-methyl-1H-indazol-5-yl)methyl]benzenesulfonamide (10). White solid (15 mg, 36%): m.p. 148-150° C.; purification (hexanes: EtOAc, 60:40), R_{f}=0.19. ¹H NMR (400 MHz, CDCl₃): δ=7.91 (d, J=0.8 Hz, 1H), 7.79 (dt, J=8.6 Hz, J=2.0 (×2) Hz, 2H), 7.53-7.51 (m, 1H), 7.45 (dt, J=8.6 Hz, J=2.0 (×2) Hz, 2H), 7.31 (d, J=8.6 Hz, 1H), 7.23 (dd, J=8.6 Hz, J=1.6 Hz, 1H), 4.69 (bt, J=6.3 Hz, 1H), 4.26 (d, J=6.3 Hz, 2H), 4.06 (s, 3H) ppm. ¹³C NMR (100 MHz, CDCl₃): δ=139.5, 139.2, 138.6, 132.7, 129.4, 128.6, 128.0, 126.5, 123.9, 120.5, 109.5, 47.6, 35.7 ppm. IR (neat): v=3300, 2923, 2851, 1514, 1326, 1150, 1091, 817, 750, 620 cm⁻¹. HRMS (ESI): calculated for C₁₅H₁₅N₃O₂S₁Cl₁ [M+H]⁺ requires m/z 336.05735, found m/z 336.05640.
4-chloro-N-[(1-methyl-1H-pyrazol-4-yl)methyl]benzenesulfonamide (11). White solid (19 mg, 51%): m.p. 125-127° C.; purification (hexanes: EtOAc, 60:40), R_{f}=0.06. ¹H NMR (400 MHz, CDCl₃): δ=7.78 (dt, J=8.6 Hz, J=2.0 (×2) Hz, 2H), 7.48 (dt, J=8.6 Hz, J=2.0 (×2) Hz, 2H), 7.24 (s, 1H), 7.20 (s, 1H), 4.90 (bt, J=5.5 Hz, 1H), 4.03 (d, J=5.5 Hz, 2H), 3.81 (s, 3H). ¹³C NMR (100 MHz, CDCl₃): δ=139.2, 138.6, 138.5, 129.5, 129.4, 128.6, 116.7, 39.0, 37.8 ppm. IR (neat): v=3148, 3054, 2936, 2856, 2782, 1741, 1585, 1566, 1473, 1340, 1159, 1059, 997, 846, 758, 612 cm⁻¹. HRMS (ESI): calculated for C₁₁H₁₃Cl₁N₃O₂S₁ [M+H]⁺ requires m/z 286.04170, found m/z 286.03979.
N-[(2-methoxypyridin-3-yl)methyl]-4-methylbenzenesulfonamide (12). Additional purification (after column chromatography) included dissolving the mixture of product amine and sulfonamide starting material in 5 mL of EtOAc along with 5 mL of 5M HCl in a separatory funnel. The aqueous acid (containing the protonated pyridinium product) was separated from the organic and basified with approximately 3-4 mL of 50% w/w NaOH solution. The deprotonated product was then extracted from the aqueous portion with EtOAc (3×5 mL), dried with Na₂SO₄, and the solvent was removed under vacuum. White solid (13 mg, 36%): m.p. 95-96° C.; purification (hexanes:EtOAc, 50:50), R_{f}=0.66. ¹H NMR (400 MHz, CDCl₃): δ=8.01 (dd, J=5.1 Hz, J=2.0 Hz, 1H), 7.64 (dt, J=8.2 Hz, J=2.0 (×2) Hz, 2H), 7.37 (dd, J=7.4 Hz, J=2.0 Hz, 1H), 7.21 (d, J=7.8 Hz, 2H), 6.74 (dd, J=7.0 Hz, J=5.1 Hz, 1H), 5.15 (t, J=6.7 Hz, 1H), 4.11 (d, J=6.7 Hz, 2H), 3.88 (s, 3H), 2.39 (s, 3H) ppm. ¹³C NMR (100 MHz, CDCl₃): δ=161.4, 146.2, 143.3, 137.8, 137.2, 129.5, 127.0, 118.9, 116.7, 53.4, 43.2, 21.5 ppm. IR (neat): v=3275, 2951, 1596, 1466, 1412, 1321, 1153, 1092, 1018, 812, 776, 660 cm⁻¹. HRMS (ESI): calculated for C₁₄H₁₇N₂O₃S₁[M+H]⁺ requires m/z 293.09599, found m/z 293.09415.
N-[(2-methoxypyridin-3-yl)methyl]benzenesulfonamide (13). Additional purification (after column chromatography) included dissolving the mixture of product amine and sulfonamide starting material in 5 mL of EtOAc along with 5 mL of 5M HCl in a separatory funnel. The aqueous acid (containing the protonated pyridinium product) was separated from the organic and basified with approximately 3-4 mL of 50% w/w NaOH solution. The deprotonated product was then extracted from the aqueous portion with EtOAc (3×5 mL), dried with Na₂SO₄, and the solvent was removed under vacuum. White solid (12 mg, 32%): m.p. 116-118° C.; purification (hexanes: EtOAc, 50:50), R_{f}=0.47. ¹H NMR (400 MHz, CDCl₃): δ=7.97 (d, J=5.1 Hz, 1H), 7.75 (m, 2H), 7.42-7.34 (m, 3H), 6.71 (dd, J=7.0 Hz, 5.1 Hz, 1H), 5.40 (bs, 1H), 4.12 (d, J=6.3 Hz, 2H), 3.84 (s, 3H) ppm. ¹³C NMR (100 MHz, CDCl₃): δ=161.3, 146.2, 140.1, 137.7, 132.5, 128.8, 126.8, 118.7, 116.7, 53.3, 43.1 ppm. IR (neat): v=3057, 2917, 2850, 1601, 1589, 1468, 1411, 1363, 1334, 1250, 1158, 1080, 1012, 105, 749, 693, 579, 528 cm⁻¹. HRMS (ESI): calculated for C₁₃H₁₅N₂O₃S₁ [M+H]c⁺ requires m/z 279.08034, found m/z 279.07294.
4-chloro-N-(pyrimidin-5-ylmethyl)benzenesulfonamide (14). White solid (9 mg, 26%): m.p. 154-160° C.; purification (100% EtOAc), R_{f}=0.46; ¹H NMR (400 MHz, CDCl₃): δ=9.14 (s, 1H), 8.64 (s, 2H), 7.79 (dt, J=8.2 Hz, J=2.7 (×2) Hz, 2H), 7.51 (dt, J=8.2 Hz, J=2.7 (×2) Hz, 2H), 5.01 (bs, 1H), 4.22 (s, 2H) ppm. ¹³C NMR (100 MHz, CDCl₃): δ=158.3, 156.5, 139.8, 138.0, 130.0, 129.7, 128.5, 42.5 ppm. IR (neat): v=3047, 2852, 1567, 1411, 1321, 1155, 822, 722, 608 cm⁻¹. HRMS (ESI): calculated for C₁₁H₁₁N₃O₂S₁Cl₁ [M+H]⁺ requires m/z 284.02605, found m/z 284.02499.
N-(pyrimidin-5-ylmethyl)benzenesulfonamide (15). White solid (8 mg, 26%): m.p. 54-60° C.; purification (100% EtOAc), R_{f}=0.34. ¹H NMR (400 MHz, CDCl₃): δ=9.11 (s, 1H), 8.62 (s, 2H), 7.88-7.84 (m, 2H), 7.64-7.59 (m, 1H), 7.56-7.51 (m, 2H), 5.11 (bs, 1H), 4.22 (s, 2H) ppm. ¹³C NMR (100 MHz, CDCl₃): δ=158.3, 156.4, 139.5, 133.2, 130.1, 129.4, 127.0, 42.5 ppm. IR (neat): v=3084, 2876, 1675, 1569, 1445, 1410, 1313, 1154, 1074, 1043, 688 cm⁻¹. HRMS (ESI): calculated for C₁₁H₁₂N₃O₂S₁ [M+H]⁺ requires m/z 250.06502, found m/z 250.06349.
4-methyl-N-(pyrazin-2-ylmethyl)benzenesulfonamide (16). White solid (14 mg, 42%): m.p. 87-92° C.; purification (100% EtOAc), R_{f}=0.54. ¹H NMR (400 MHz, CDCl₃): δ=8.50-8.41 (m, 3H), 7.74 (d, J=8.2 Hz, 2H), 7.26 (d, J=8.2 Hz, 2H), 5.56 (bt, J=5.5 Hz, 1H), 4.32 (d, J=5.5 Hz, 2H), 2.40 (s, 3H) ppm. ¹³C NMR (100 MHz, CDCl₃): δ=143.9, 143.8, 143.7, 143.6, 136.4, 129.8, 129.7, 127.2, 45.5, 21.5 ppm. IR (neat): v=3130, 2916, 1599, 1458, 1406, 1329, 1187, 1091, 1018, 870, 816, 707, 663 cm⁻¹. HRMS (ESI): calculated for C₁₂H₁₄N₃O₂S₁ [M+H]⁺ requires m/z 264.08067, found m/z 264.07947.
4-methyl-N-(1,3-thiazol-4-ylmethyl)benzenesulfonamide (17). White solid (15 mg, 44%): m.p. 119-122° C.; purification (hexanes:EtOAc, 60:40), R_{f}=0.18. ¹H NMR (400 MHz, CDCl₃): δ=8.68 (d, J=2.0 Hz, 1H), 7.67 (dt, J=8.2 Hz, J=2.0 (×2) Hz, 2H), 7.25-7.21 (m, 2H), 7.11-7.09 (m, 1H), 5.65 (bt, J=6.3 Hz, 1H), 4.32 (d, J=6.3 Hz, 2H), 2.39 (s, 3H) ppm. ¹³C NMR (100 MHz, CDCl₃): δ=153.6, 152.3, 143.4, 136.9, 129.6, 127.1, 115.7, 42.9, 21.5 ppm. IR (neat): v=3070, 2863, 1600, 1458, 1412, 1314, 1258, 1146, 1093, 1072, 812, 730, 662 cm⁻¹. HRMS (ESI): calculated for C₁₁H₁₃N₂O₂S₂ [M+H]⁺ requires m/z 269.04185, found m/z 269.04280.
4-chloro-N-(1,3-thiazol-4-ylmethyl)benzenesulfonamide (18). White solid (13 mg, 35%): m.p. 130-132° C.; purification (100% EtOAc), R_{f}=0.86. ¹H NMR (400 MHz, CDCl₃): δ=8.68 (d, J=2.3 Hz, 1H), 7.70 (dt, J=9.0 Hz, J=2.4 (×2) Hz, 2H), 7.39 (dt, J=9.0 Hz, J=2.4 (×2) Hz, 2H), 7.10 (m, 1H), 5.82 (bt, J=6.3 Hz, 1H), 4.35 (d, J=6.3 Hz, 2H) ppm. ¹³C NMR (100 MHz, CDCl₃): δ=153.8, 152.0, 139.0, 138.5, 129.2, 128.5, 116.0, 42.9 ppm. IR (neat): v=3257, 3109, 1586, 1476, 1325, 1313, 1278, 1158, 1092, 1047, 933, 878, 828, 756, 662, 615, 507 cm⁻¹. HRMS (ESI): calculated for C₁₀H₁₀C₁₁N₂O₂S₂ [M+H]⁺ requires m/z 288.98722, found m/z 288.97977.
4-methyl-N-(1,3-thiazol-2-ylmethyl)benzenesulfonamide (19). Off-white oil (17 mg, 51%): purification (hexanes:EtOAc, 60:40), R_{f}=0.15. ¹H NMR (400 MHz, CDCl₃): δ=7.76 (dt, J=8.2 Hz, J=2.0 (×2) Hz, 2H), 7.65 (d, J=3.1 Hz, 1H), 7.31-7.25 (m, 3H), 5.56 (bt, J=6.3 Hz, 1H), 4.48 (d, J=6.3 Hz, 2H), 2.42 (s, 3H) ppm. ¹³C NMR (100 MHz, CDCl₃): δ=165.9, 143.8, 142.5, 136.5, 129.8, 127.2, 119.9, 44.4, 21.5 ppm. IR (neat): v=3084, 2850, 1504, 1329, 1159, 1090, 1058, 736, 658 cm⁻¹. HRMS (ESI): calculated for C₁₁H₁₃N₂O₂S₂[M+H]⁺ requires m/z 269.04185, found m/z 269.04013.
4-chloro-N-(furan-2-ylmethyl)benzenesulfonamide (20). White solid (18 mg, 52%): m.p. 118-120° C.; purification (hexanes:EtOAc, 60:40), R_{f}=0.65. ¹H NMR (400 MHz, CDCl₃): δ=7.75 (dt, J=9.0 Hz, J=2.4 (×2) Hz, 2H), 7.44 (dt, J=9.0 Hz, J=2.4 (×2) Hz, 2H), 7.23 (m, 1H), 6.22 (dd, J=3.3 Hz, J=1.8 Hz, 1H), 6.10 (m, 1H), 4.79 (bt, J=5.9 Hz, 1H), 4.22 (d, J=5.9 Hz, 2H) ppm. ¹³C NMR (100 MHz, CDCl₃): δ=149.1, 142.6, 139.1, 138.5, 129.2, 128.5, 110.4, 108.5, 40.1 ppm. IR (neat): v=3260, 1586, 1476, 1433, 1320, 1158, 1148, 1091, 1012, 922, 881, 824, 724 cm⁻¹. HRMS (ESI): calculated for C₁₁H₁₁N₁O₃S₁Cl₁ [M+H]⁺ requires m/z 272.01482, found m/z 272.01401.
4-methyl-N-[(2-methyl-1,3-oxazol-4-yl)methyl]benzenesulfonamide (21). White solid (19 mg, 57%): m.p. 156-158° C.; purification (hexanes:EtOAc, 60:40), R_{f}=0.06. ¹H NMR (400 MHz, CDCl₃): δ=7.71 (dt, J=8.2 Hz, J=2.0 (×2) Hz, 2H), 7.31 (t, J=1.2 Hz, 1H), 7.27 (d, J=8.2 Hz, 2H), 5.14 (bt, J=6.3 Hz, 1H), 4.03 (dd, J=6.3 Hz, J=1.2 Hz, 2H), 2.42 (s, 3H), 2.35 (s, 3H) ppm. ¹³C NMR (100 MHz, CDCl₃): δ=162.0, 143.5, 136.7, 135.9, 135.2, 129.6, 127.2, 39.1, 21.5, 13.7 ppm. IR (neat): v=3102, 2959, 2928, 2873, 1725, 1578, 1461, 1322, 1275, 1153, 1072, 930, 766, 660 cm⁻¹. HRMS (ESI): calculated for C₁₂H₁₅N₂O₃S₁ [M+H]⁺ requires m/z 267.08034, found m/z 267.07907.
4-methyl-N-(quinolin-6-ylmethyl)benzenesulfonamide (22). White solid (12 mg, 31%). m.p. 148-150° C. Purification (EtOAc). R_{f}=0.51. ¹H NMR (400 MHz, CDCl₃): δ=8.90 (dd, J=4.1 Hz, J=1.8 Hz, 1H), 8.07 (dd, J=7.8 Hz, J=1.2 Hz, 1H), 8.01 (d, J=8.6 Hz, 1H), 7.77 (m, 2H), 7.65 (d, J=1.2 Hz, 1H), 7.52, (dd, J=8.6 Hz, J=2.2 Hz, 1H), 7.40 (dd, J=8.6 Hz, J=4.1 Hz, 1H), 7.29 (m, 2H), 4.84 (bt, J=6.3 Hz, 1H), 4.34 (d, J=6.3 Hz, 2H), 2.41 (s, 3H) ppm. ¹³C NMR (100 MHz, CDCl₃) δ 150.6, 147.7, 143.7, 136.9, 135.9, 134.7, 129.9, 129.8, 129.2, 128.0, 127.2, 126.4, 121.5, 47.0, 21.5 ppm. IR (neat): v=3097, 2924, 2841, 1595, 1313, 1152, 885, 837, 658, 540 cm⁻¹. HRMS (ESI): calculated for C₁₇H₁₇N₂SiO₂ [M+H]⁺ requires m/z 313.10108, found m/z 313.10080.
N-(1H-indol-3-ylmethyl)-4-methoxybenzenesulfonamide (23). Pale yellow solid (25 mg, 63%). m.p. 170-173° C. Purification (hexanes: EtOAc, 50:50). R_{f}=0.43. ¹H NMR (400 MHz, (CD₃)₂CO) δ=10.08 (bs, 1H), 7.84 (dt, J=8.6 Hz, J=3.1 Hz, 2H), 7.51 (d, J=7.8 Hz, 1H), 7.36 (d, J=7.8 Hz, 1H), 7.18 (d, J=2.0 Hz, 1H), 7.09 (m, 3H), 6.99 (m, 1H), 6.42 (bt, J=6.3 (×2) Hz, 1H), 4.26 (d, J=6.3 Hz, 2H), 3.90 (s, 3H) ppm. ¹³C NMR (100 MHz, (CD₃)₂CO): δ=163.5, 137.7, 133.7, 130.0, 127.7, 124.8, 122.4, 119.8, 119.6, 114.9, 112.2, 111.8, 56.0, 39.6 ppm. IR (neat): v=3385, 3290, 3003, 2837, 1594, 1261, 1154, 1022, 538 cm⁻¹. HRMS (ESI): calculated for C₁₆H₁₆N₂S₁O₃[M+Na]⁺ requires m/z 339.07794, found m/z 339.07790.
N-(1H-indol-3-ylmethyl)-4-(trifluoromethyl)benzenesulfonamide (24). White solid (18 mg, 41%). m.p. 199-201° C. Purification (hexanes: EtOAc, 60:40). R_{f}=0.37. ¹H NMR (400 MHz, (CD₃)₂CO): δ=10.08 (bs, 1H), 8.01 (d, J=8.6 Hz, 2H), 7.79 (d, J=8.2 Hz, 2H), 7.49 (d, J=7.8 Hz, 1H), 7.31 (dd, J=8.2 Hz, J=0.78 Hz, 1H), 7.19 (d, J=1.6 Hz, 1H), 7.08 (t, J=7.6 (×2) Hz, 1H), 6.97 (m, 2H), 4.38 (d, J=5.9 Hz, 2H) ppm. ¹³C NMR (100 MHz, (CD₃)₂CO): δ 146.0, 137.6, 133.7, 133.4, 128.5, 127.5, 126.6, 125.2, 122.5, 119.8, 119.4, 112.2, 111.4, 39.7 ppm. IR (neat): v=3394, 3312, 1404, 1358, 1158, 846, 744 cm⁻¹. HRMS (ESI): calculated for C₁₆H₁₃N₂S₁O₂F₃ [M+Na]⁺ requires m/z 377.05475, found m/z 377.05430.
N-(1H-indol-3-ylmethyl)-3-nitrobenzenesulfonamide (25). Yellow solid (8 mg, 19%). m.p. 182-184° C. Purification (hexanes: EtOAc, 50:50). R_{f}=0.46. ¹H NMR (400 MHz, (CD₃)₂CO): δ=10.05 (bs, 1H), 8.40 (m, 1H), 8.20 (dt, J=8.2 Hz, J=0.78 Hz, 1H), 8.05 (dt, J=7.8 Hz, J=0.78 Hz, 1H), 7.60 (t, J=8.0 (×2) Hz, 1H), 7.48 (d, J=7.8 Hz, 1H), 7.26-7.17 (m, 3H), 7.01 (t, J=7.6 (×2) Hz, 1H), 6.90 (m, 1H), 4.43 (d, J=5.9 Hz, 2H) ppm. ¹³C NMR (100 MHz, (CD₃)₂CO): δ=144.0, 137.5, 133.2, 130.8, 127.3, 126.8, 125.5, 125.3, 122.5, 122.4, 119.8, 119.4, 112.0, 111.3, 39.7 ppm. IR (neat): v=3386, 3303, 3105, 2923, 1522, 1346, 1159, 748 cm⁻¹. HRMS (ESI): calculated for C₁₅H₁₄N₃SiO₄ [M+Na]⁺ requires m/z 354.05245, found m/z 354.05190.
N-(1H-indol-3-ylmethyl)methanesulfonamide (26). Brown solid (9 mg, 34%). m.p. 133-135° C. Purification (hexanes: EtOAc, 50:50). R_{f}=0.28. ¹H NMR (400 MHz, (CD₃)₂CO): δ=10.20 (bs, 1H), 7.73 (d, J=7.8 Hz, 1H), 7.42 (d, J=7.8 Hz, 1H), 7.36 (d, J=2.0 Hz, 1H), 7.13 (t, J=7.4 (×2) Hz, 1H), 7.06 (t, J=7.4 (×2) Hz, 1H), 6.20 (bs, 1H), 4.49 (d, J=5.9 Hz, 2H), 2.82 (s, 3H) ppm. ¹³C NMR (100 MHz, (CD₃)₂CO): δ=137.8, 127.7, 124.9, 122.5, 119.9, 119.6, 112.4, 112.3, 40.3, 39.5 ppm. IR (neat): v=3394, 3270, 3016, 2929, 2527, 1140, 738, 505, 428 cm⁻¹. HRMS (ESI): calculated for C₁₀H₁₂N₂S₁O₂ [M+Na]⁺ requires m/z 247.05172, found m/z 247.05120.
N-[(4-bromo-1H-indol-3-yl)methyl]-4-chlorobenzenesulfonamide (27). White solid (7 mg, 25%). M.p. 186-188° C. Purification (hexanes: EtOAc, 50:50). R_{f}=0.53. ¹H NMR (400 MHz, (CD₃)₂CO) δ=10.48 (bs, 1H), 7.83 (dt, J₁=8.6 Hz, 12=2.3 Hz, 2H), 7.51 (dt, J₁=8.2 Hz, J₂=2.3 Hz, 2H), 7.38 (d, J₁=8.2 Hz, 1H), 7.34 (d, J₁=1.6 Hz, 1H), 7.16 (d, J₁=7.4 Hz, 1H), 6.98 (t, J₁=7.4×(2) Hz, 1H), 6.61 (t, J₁=5.5×(2) Hz, 1H), 4.55 (d, J₁=5.1 Hz, 2H) ppm. ¹³C NMR (100 MHz, (CD₃)₂CO) δ 140.2, 138.1, 128.8, 128.72, 128.70, 126.9, 124.9, 123.2, 122.5, 112.9, 111.12, 111.07, 39.1 ppm. IR (neat): v=3355, 1704, 1334, 1189, 1092, 747 cm⁻¹. HRMS (ESI): calculated for C₁₅H₁₁BrClN₂O₂S [M−H]⁺ requires m/z 396.94131, found m/z 396.94183.
4-chloro-N-[(7-methyl-1H-indol-3-yl)methyl]benzenesulfonamide (28). Tan solid (17 mg, 42%). M.p. 204-206° C. Purification (hexanes: EtOAc, 50:50). R_{f}=0.59. ¹H NMR (400 MHz, (CD₃)₂CO) δ=10.06 (bs, 1H), 7.81 (dt, J₁=9.0 Hz, J₂=2.3 Hz, 2H), 7.51 (dt, J₁=10.2 Hz, J₂=3.1 Hz, 2H), 7.34 (t, J₁=4.5×(2) Hz, 1H), 7.16 (d, J₁=2.0 Hz, 1H), 6.90 (d, J₁=4.7 Hz, 2H), 6.76 (t, J₁=4.7×(2) Hz, 1H), 4.32 (d, J₁=5.9 Hz, 2H), 2.45 (s, 3H) ppm. ¹³C NMR (100 MHz, (CD₃)₂CO) δ 140.1, 137.4, 136.2, 128.7, 128.6, 126.3, 123.8, 122.1, 120.5, 119.2, 116.3, 111.0, 38.9, 15.9 ppm. IR (neat): v=3402, 3282, 3093, 2917, 1318, 1158, 1093, 1024, 484 cm⁻¹. HRMS (ESI): calculated for C₁₆H₁₄ClN₂O₂S [M−H]⁺ requires m/z 333.04645, found m/z 333.04688.
4-chloro-N-[(1-methyl-1H-indol-3-yl)methyl]benzenesulfonamide (29). White solid (14 mg, 32%). m.p. 184-187° C. Purification (hexanes: EtOAc, 50:50). R_{f}=0.70. ¹H NMR (400 MHz, (CDCl₃): δ=7.75 (dt, J=9.4 Hz, J=2.7 Hz, 2H), 7.43-7.36 (m, 3H), 7.28-7.21 (m, 2H), 7.08 (ddd, J=8.0 Hz, J=6.5 Hz, J=1.6 Hz, 1H), 6.86 (s, 1H), 4.61 (bt, J=5.5 (×2) Hz, 1H), 4.33 (d, J=5.5 Hz, 2H), 3.70 (s, 3H) ppm. ¹³C NMR (100 MHz, (CDCl₃): δ=138.9, 138.5, 137.1, 129.1, 128.6, 128.1, 126.5, 122.3, 119.7, 118.5, 109.5, 108.9, 38.9, 32.7 ppm. IR (neat): v=3303, 3058, 1473, 1311, 1157, 1041, 826, 736, 544 cm⁻¹. HRMS (ESI): calculated for C₁₆H₁₅N₂S₁O₂Cl₁ [M+Na]⁺ requires m/z 357.04405, found m/z 357.04210.
4-chloro-N-[(4-methyl-1H-indol-3-yl)methyl]benzenesulfonamide (30). Tan solid (16 mg, 37%). m.p. 160-162° C. Purification (hexanes: EtOAc, 60:40). R_{f}=0.43. ¹H NMR (400 MHz, (CD₃)₂CO): δ=10.12 (bs, 1H), 7.91 (dt, J=9.0 Hz, J=2.0 Hz, 2H), 7.61 (dt, J=9.0 Hz, J=2.0 Hz, 2H), 7.18 (d, J=8.2 Hz, 1H), 7.15 (d, J=2.3 Hz, 1H), 6.96 (dd, J=8.2, J=7.0 Hz, 1H), 6.74 (dt, J=7.0 Hz, J=1.0 (×2) 1H), 6.62 (bt, J=5.5 Hz, 1H), 4.38 (d, J=5.5 Hz, 2H), 2.57 (s, 3H) ppm. ¹³C NMR (100 MHz, (CD₃)₂CO): δ=140.7, 138.6, 138.1, 130.9, 129.9, 129.7, 126.3, 126.0, 122.6, 121.5, 111.6, 110.2, 41.2, 20.1 ppm. IR (neat): v=3390, 3273, 2923, 1336, 1150, 1093, 752 cm⁻¹. HRMS (ESI): calculated for C₁₆H₁₆Cl₁N₂O₂S₁ [M+H]⁺ requires m/z 335.0621, found m/z 335.0610.
Each of the foregoing and following compounds are depicted in FIGS. 1A-1L as identified by the number in parentheses following the name of the compound.
4-methyl-N-[(2-methyl-1H-indol-3-yl)methyl]benzenesulfonamide (31)
4-chloro-N-[(2-methyl-1H-indol-3-yl)methyl]benzenesulfonamide (32)
4-chloro-N-[(2-chloro-1H-indol-3-yl)methyl]benzenesulfonamide (33)
4-chloro-N-[(5-methoxy-1H-indol-3-yl)methyl]benzenesulfonamide (34)
4-chloro-N-[(5-fluoro-1H-indol-3-yl)methyl]benzenesulfonamide (35)
4-chloro-N-[(5-nitro-1H-indol-3-yl)methyl]benzenesulfonamide (36)
4-fluoro-N-(1H-indol-3-ylmethyl)benzenesulfonamide (37)
4-bromo-N-(1H-indol-3-ylmethyl)benzenesulfonamide (38)
4-chloro-N-(quinolin-6-ylmethyl)benzenesulfonamide (39)
3-bromo-N-(1H-indol-3-ylmethyl)benzenesulfonamide (40)
4-chloro-N-(1H-indol-4-ylmethyl)benzenesulfonamide (41)
4-chloro-N-(1H-indol-5-ylmethyl)benzenesulfonamide (42)
4-chloro-N-(1H-indol-6-ylmethyl)benzenesulfonamide (43)
4-chloro-N-{[1-(phenylsulfonyl)-H-indol-2-yl]methyl}benzenesulfonamide (44)
3-chloro-N-(1H-indol-3-ylmethyl)benzenesulfonamide (45)
3-fluoro-N-(1H-indol-3-ylmethyl)benzenesulfonamide (46)
2-fluoro-N-(1H-indol-3-ylmethyl)benzenesulfonamide (47)
2-bromo-N-(1H-indol-3-ylmethyl)benzenesulfonamide (48)
N-(1H-indol-3-ylmethyl)-2-methylbenzenesulfonamide (49)
3,5-dichloro-N-(1H-indol-3-ylmethyl)benzenesulfonamide (50)
3,5-difluoro-N-(1H-indol-3-ylmethyl)benzenesulfonamide (51)
2,3-dichloro-N-(1H-indol-3-ylmethyl)benzenesulfonamide (52)
2,4-difluoro-N-(1H-indol-3-ylmethyl)benzenesulfonamide (53)
N-(1H-indol-3-ylmethyl)-2-nitrobenzenesulfonamide (54)
N-(1H-indol-6-ylmethyl)pyridine-3-sulfonamide (55)
N-(1H-indol-6-ylmethyl)biphenyl-4-sulfonamide (56)
5-chloro-N-(1H-indol-6-ylmethyl)thiophene-2-sulfonamide (57)
N-(1H-indol-5-ylmethyl)-4-methylbenzenesulfonamide (58)
4-bromo-N-(1H-indol-6-ylmethyl)benzenesulfonamide (59)
N-(1H-indol-6-ylmethyl)-4-methoxybenzenesulfonamide (60)
N-(1H-indol-6-ylmethyl)-4-phenoxybenzenesulfonamide (61)
4-fluoro-N-(1H-indol-6-ylmethyl)benzenesulfonamide (62)
N-(1H-indol-6-ylmethyl)-4-nitrobenzenesulfonamide (63)
N-(1H-indol-6-ylmethyl)-4-(trifluoromethyl)benzenesulfonamide (64)
N-(1H-indol-6-ylmethyl)-4-methylbenzenesulfonamide (65)
3-chloro-N-(1H-indol-6-ylmethyl)benzenesulfonamide (66)
3-fluoro-N-(1H-indol-6-ylmethyl)benzenesulfonamide (67)
N-(1H-indol-6-ylmethyl)-3-(trifluoromethyl)benzenesulfonamide (68)
N-(1H-indol-6-ylmethyl)-3-nitrobenzenesulfonamide (69)
2-chloro-N-(1H-indol-6-ylmethyl)benzenesulfonamide (70)
2-fluoro-N-(1H-indol-6-ylmethyl)benzenesulfonamide (71)
N-(1H-indol-6-ylmethyl)-2-(trifluoromethyl)benzenesulfonamide (72)
N-(1H-indol-6-ylmethyl)-2-nitrobenzenesulfonamide (73)
2,4-dichloro-N-(1H-indol-6-ylmethyl)benzenesulfonamide (74)
3,5-dichloro-N-(1H-indol-6-ylmethyl)benzenesulfonamide (75)
2,6-difluoro-N-(1H-indol-6-ylmethyl)benzenesulfonamide (76)
4-fluoro-N-(1H-indol-6-ylmethyl)-2-methylbenzenesulfonamide (77)
5-fluoro-N-(1H-indol-6-ylmethyl)-2-methylbenzenesulfonamide (78)
N-(1H-indol-6-ylmethyl)thiophene-2-sulfonamide (79)
N-(1H-indol-6-ylmethyl)quinoline-8-sulfonamide (80)
N-(1H-indol-6-ylmethyl)morpholine-4-sulfonamide (81)
4-bromo-N-(1H-indol-5-ylmethyl)benzenesulfonamide (82)
N-(1H-indol-5-ylmethyl)biphenyl-4-sulfonamide (83)
N-(1H-indol-5-ylmethyl)-4-phenoxybenzenesulfonamide (84)
N-(1H-indol-5-ylmethyl)-4-methoxybenzenesulfonamide (85)
N-(1H-indol-5-ylmethyl)-4-nitrobenzenesulfonamide (86)
N-(1H-indol-5-ylmethyl)-4-(trifluoromethyl)benzenesulfonamide (87)
4-fluoro-N-(1H-indol-5-ylmethyl)benzenesulfonamide (88)
N-(1H-indol-5-ylmethyl)-3-nitrobenzenesulfonamide (89)
N-(1H-indol-5-ylmethyl)-3-(trifluoromethyl)benzenesulfonamide (90)
3-fluoro-N-(1H-indol-5-ylmethyl)benzenesulfonamide (91)
3-chloro-N-(1H-indol-5-ylmethyl)benzenesulfonamide (92)
N-(1H-indol-5-ylmethyl)-2-nitrobenzenesulfonamide (93)
N-(1H-indol-5-ylmethyl)-2-(trifluoromethyl)benzenesulfonamide (94)
2-fluoro-N-(1H-indol-5-ylmethyl)benzenesulfonamide (95)
2-chloro-N-(1H-indol-5-ylmethyl)benzenesulfonamide (96)
2,4-dichloro-N-(1H-indol-5-ylmethyl)benzenesulfonamide (97)
3,5-dichloro-N-(1H-indol-5-ylmethyl)benzenesulfonamide (98)
2,6-difluoro-N-(1H-indol-5-ylmethyl)benzenesulfonamide (99)
4-fluoro-N-(1H-indol-5-ylmethyl)-2-methylbenzenesulfonamide (100)
5-fluoro-N-(1H-indol-5-ylmethyl)-2-methylbenzenesulfonamide (101)
N-(1H-indol-5-ylmethyl)thiophene-2-sulfonamide (102)
5-chloro-N-(1H-indol-5-ylmethyl)thiophene-2-sulfonamide (103)
N-(1H-indol-5-ylmethyl)pyridine-3-sulfonamide (104)
N-(1H-indol-5-ylmethyl)quinoline-8-sulfonamide (105)
N-(1H-indol-5-ylmethyl)morpholine-4-sulfonamide (106)
N-(1H-indol-5-ylmethyl)-4-methylpiperidine-1-sulfonamide (107)
N-(1H-indol-4-ylmethyl)-4-methylbenzenesulfonamide (108)
4-bromo-N-(1H-indol-4-ylmethyl)benzenesulfonamide (109)
N-(1H-indol-4-ylmethyl)biphenyl-4-sulfonamide (110)
N-(1H-indol-4-ylmethyl)-4-phenoxybenzenesulfonamide (111)
N-(1H-indol-4-ylmethyl)-4-nitrobenzenesulfonamide (112)
N-(1H-indol-4-ylmethyl)-4-(trifluoromethyl)benzenesulfonamide (113)
N-(1H-indol-4-ylmethyl)-4-methoxybenzenesulfonamide (114)
4-fluoro-N-(1H-indol-4-ylmethyl)benzenesulfonamide (115)
3-fluoro-N-(1H-indol-4-ylmethyl)benzenesulfonamide (116)
N-(1H-indol-4-ylmethyl)-3-(trifluoromethyl)benzenesulfonamide (117)
N-(11H-indol-4-ylmethyl)-3-nitrobenzenesulfonamide (118)
3-chloro-N-(1H-indol-4-ylmethyl)benzenesulfonamide (119)
N-(1H-indol-4-ylmethyl)benzenesulfonamide (120)
2-fluoro-N-(1H-indol-4-ylmethyl)benzenesulfonamide (121)
N-(11H-indol-4-ylmethyl)-2-(trifluoromethyl)benzenesulfonamide (122)
N-(1H-indol-4-ylmethyl)-2-nitrobenzenesulfonamide (123)
2-chloro-N-(1H-indol-4-ylmethyl)benzenesulfonamide (124)
2,4-dichloro-N-(1H-indol-4-ylmethyl)benzenesulfonamide (125)
3,5-dichloro-N-(1H-indol-4-ylmethyl)benzenesulfonamide (126)
2,6-difluoro-N-(1H-indol-4-ylmethyl)benzenesulfonamide (127)
4-fluoro-N-(1H-indol-4-ylmethyl)-2-methylbenzenesulfonamide (128)
5-fluoro-N-(1H-indol-4-ylmethyl)-2-methylbenzenesulfonamide (129)
N-(1H-indol-4-ylmethyl)thiophene-2-sulfonamide (130)
5-chloro-N-(1H-indol-4-ylmethyl)thiophene-2-sulfonamide (131)
N-(1H-indol-4-ylmethyl)pyridine-3-sulfonamide (132)
N-(1H-indol-4-ylmethyl)quinoline-8-sulfonamide (133)
N-(1H-indol-4-ylmethyl)-2-methylbenzenesulfonamide (134)
N-(1H-indol-4-ylmethyl)-2-(trifluoromethoxy)benzenesulfonamide (135)
2-bromo-N-(1H-indol-4-ylmethyl)benzenesulfonamide (136)
3-bromo-N-(1H-indol-4-ylmethyl)benzenesulfonamide (137)
2,3-dichloro-N-(1H-indol-4-ylmethyl)benzenesulfonamide (138)
2,4,6-trichloro-N-(1H-indol-4-ylmethyl)benzenesulfonamide (139)

### Method of In Vitro Screening for Pharmacological Activity Against a Patient's Specific Type of Cancer

The present disclosure provides a method for determining whether or not a variety of compounds will likely have pharmacological activity against a patient's specific cancer cells. The following methods were developed to assess the in vitro effectiveness of the compounds alone, when combined with a metabolic inhibitor or in media formulated to promote use of specific metabolic pathways. However, prior to carrying out the screening method to determine the pharmacological effectiveness of a compound, patient cancer cells must first be obtained and transported to the testing facility.

Cancer cells must acquire nutrients, growth factors, and other components from the patient's own circulatory system, and so have evolved and are adapted to the unique blood serum in which they grow. Therefore, to provide the most accurate assessment of the potential for a compound to treat cancer, the compounds must be tested on cancerous cells obtained from the patient. Cancer cells can be obtained from the patient through conventional biopsy practices, including but not limited to resected tumor, needle biopsy and blood in the case of hematopoietic cancers. These cells are added to a patient derived serum and/or plasma. The patient derived serum and/or plasma having previously been prepared. As used herein, the terms “patient serum” and “patient plasma” indicates that the serum and/or plasma was obtained from the patient. Serum and plasma both come from the liquid portion of the blood that remains once the cells are removed. However, serum is the liquid that remains after the blood has clotted, while plasma is the liquid that remains when clotting is prevented with the addition of an anticoagulant. The following methods may use patient serum, patient plasma or a combination of both. The final choice will be made by the individual conducting the screening method based on the patient status and the cancer type.

The patient derived serum and/or plasma is one optional base media use for preparing patient derived cancer cells in individualized media. In this option, 100% of the base media is patient derived serum and/or plasma. Another optional base media is a conventional cell base media designed to mimic the composition and in vivo environment of the organ in which the cancer appears. The conventional base media is supplemented with between about 5% to about 25% of the patient derived serum and/or plasma.

The method of preparing the patient derived cancer cells includes the use of individualized media specifically formulated to correspond to the location of the cancer within the patient's body, i.e. individualized cancer cell support media. The individualized cancer cell support media includes the patient's cancer cells and either a base media of 100% patient derived serum and/or plasma or the above described conventional cell base media with about 5% to about 25% of the patient derived serum and/or plasma. The individualized cancer cell support media simulates the in vivo environment by inclusion of amino acids, vitamins, inorganic salts and glucose in concentrations corresponding to the concentrations found in the organ in which the cancer appears. One skilled in the art will be readily able to determine the particular amino acids required and the concentrations of the amino acids and other constituents needed for the individualized media solution to which the cancer cells and patient derived serum and/or plasma will be added.

The individualized media will further include antibiotics commonly added to cell supporting individualized media, such as penicillin and streptomycin, in concentrations suitable to ensure the viability of the cells during the time necessary for transport and testing. Finally, the individualized media will include a buffering system suitable for maintaining pH of the individualized media in the range of 7.2 to 7.4. One common buffering system suitable for use in the individualized media is zwitterion HEPES. Alternatively, for long term storage or shipping, the buffering system may rely upon sodium bicarbonate and the final formulated individualized media maintained under a carbon dioxide atmosphere. Thus, the resulting individualized media corresponds closely to the patient's body chemistry, i.e. the environment and metabolic conditions of the cancer. Thus, use of the individualized media will likely result in the cultured patient-derived cancer cell metabolism remaining similar to that of cancer cells in the patient.

The patient derived serum and/or plasma and specially formulated individualized media enhance the likelihood that the harvested cancer cells will continue to use the same metabolic pathways used during cancer growth in the patient. Thus, screening of potential compounds for treatment of the cancer has a greater likelihood of identifying those compounds which will take advantage of the same metabolic pathways. Further, use of the patient derived serum and/or plasma and specially formulated individualized media enhances cell growth of the harvested cancer cells. In general, upon addition of the cancer cells to the patient derived serum and/or plasma and specially formulated individualized media, the cancer cells will immediately begin cellular reproduction. Thus, the cancer cells should not be frozen prior to shipment and/or testing. The disclosed method avoids the need to culture the cells long tenn, which has the potential to alter their metabolism such that it becomes different than that of the tumor. Additionally, the probability of cell contamination during shipping will be reduced.

By providing an environment for cancer cell growth which mimics growth within the patient, the screening method can take advantage of the same metabolic pathways. Hence the screening test will determine those compounds capable of blocking those metabolic pathways which permit cell growth of the cancer cells. Alternatively, the screening method can include steps which simulate diet and environment (e.g. sleep cycles or lack of sleep, body temperature) induced changes in the patient's body chemistry thereby resulting in a change of metabolic pathways used by the cancer cells. Following creation of the change in metabolic conditions, testing may be carried out to determine if a synergistic effect can be achieved by the combination induced metabolic changes and treatment with target compounds.

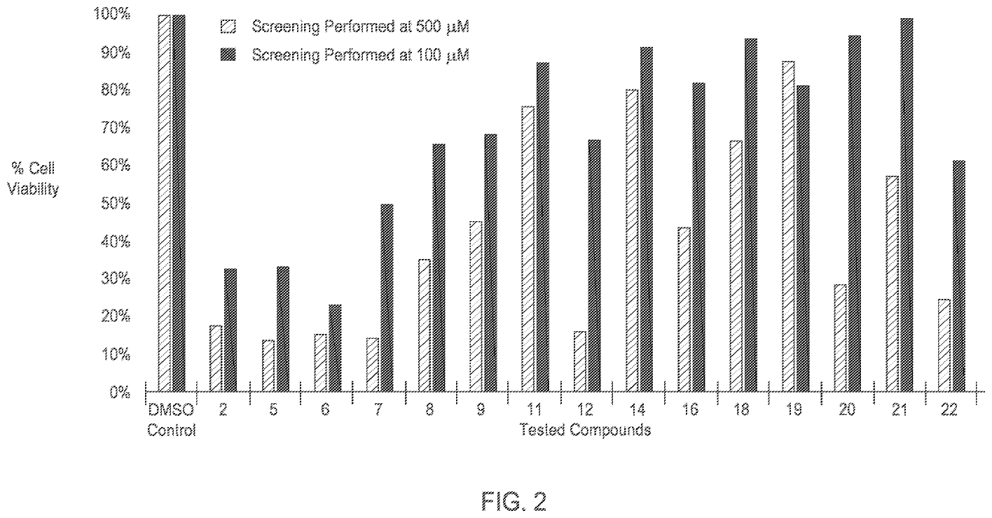

To demonstrate the ability to identify compounds active against cancer cells in vitro the N-benzyl sulfonamide library of compounds of FIGS. 1A-L was initially screened for biological activity using a standard cytotoxicity test. The cytotoxicity tests rely upon fluorescence values to determine the cytotoxic impact of the selected compounds on the selected cells. The Table of FIG. 2 demonstrates that compounds 2, 5-9, 11-12, 14, 16, 18-22 were active in cells relative to a control of DMSO. Subsequently, these compounds were tested against the following cancer cell lines obtained from American Type Culture Collection (ATCC), Manassas, VA: H293=kidney cancer; BxPC3=pancreatic cancer; HeLa=cervical cancer; MCF7, SkBr3, T47D, MDA-MB=breast cancer; MCF10A=non-cancerous breast; PC3=prostate cancer; NCI-H196=lung cancer. The compounds were also tested against normal cells (HDF).

The cytotoxicity tests were carried out in the following manner. Living cells are known to convert resazurin to the fluorescent compound resorufin. Test systems which rely upon this reaction are commercially available. One such test is known at the Cell Titer Blue Cell Viability test assay from Promega. Cell cultures for each of the identified cancer lines were obtained from ATCC and maintained in Dulbecco's Modified Eagle Medium (DMEM) supplemented with 10% fetal bovine serum (FBS) and pen/strep. As known to those skilled in the art, DMEM typically includes the components identified below.

| Ingredients | mg/L |
|---|---|
| INORGANIC SALTS | |
| Calcium chloride dihydrate | 265.000 |
| Ferric nitrate nonahydrate | 0.100 |
| Magnesium sulphate anhydrous | 97.720 |
| Potassium chloride | 400.000 |
| Sodium chloride | 6400.000 |
| AMINO ACIDS | |
| Glycine | 30.000 |
| L-Arginine hydrochloride | 84.000 |
| L-Cystine dihydrochloride | 62.570 |
| L-Glutamine | 584.000 |
| L-Histidine hydrochloride monohydrate | 42.000 |
| L-Isoleucine | 105.000 |
| L-Leucine | 105.000 |
| L-Lysine hydrochloride | 146.000 |
| L-Methionine | 30.000 |
| L-Phenylalanine | 66.000 |
| L-Serine | 42.000 |
| L-Threonine | 95.000 |
| L-Tryptophan | 16.000 |
| L-Tyrosine disodium salt | 103.790 |
| L-Valine | 94.000 |
| VITAMINS | |
| Choline chloride | 4.000 |
| D-Ca-Pantothenate | 4.000 |
| Folic acid | 4.000 |
| Nicotinamide | 4.000 |
| Pyridoxal hydrochloride | 4.000 |
| Riboflavin | 0.400 |
| Thiamine hydrochloride | 4.000 |
| i-Inositol | 7.200 |
| OTHERS | |
| D-Glucose | 4500.000 |
| Phenol red sodium salt | 15.900 |

As known to those skilled in the art, pen/strep is a combination of penicillin and streptomycin used to prevent bacterial and fungal contamination of mammalian cell cultures. The pen/strep solution contains 5,000 Units of Penicillin G (sodium salt) which acts as the active base, and 5,000 micrograms of Streptomycin (sulfate) (base per milliliter), formulated in 0.85% saline.

The test method provides for incubating the cell cultures at temperatures which correspond to the range of body temperatures experienced by the patient from which the cancer cells were obtained. Typically, incubation temperatures will be between the temperatures of 36.1° C. and 37.2° C. In most cases incubation will occur at 37° C. Additionally, the cell cultures are kept under an atmosphere which mimics cell conditions of the tumor within the patient's body. In most cases, the tumor microenvironment (TME) is characterized by hypoxia (low oxygen) and may also be characterized by hypercapnia (increased CO₂). Hypercapnia results from decreased blood flow which limits CO₂ elimination. Restricted CO₂ elimination leads to increased HCO₃⁻ conversion to CO₂ in order to neutralize lactate as altered tumor cell metabolism produces more CO₂ than normal. Normal tissue averages about 5% oxygen with oxygen ranges from about 3% to 7.4%. However, median oxygenation in untreated tumors is significantly lower, falling between approximately 0.3% and 4.2% oxygen, with most tumors exhibiting median oxygen levels<2%. In the case of CO₂, normocapnic is around 5% CO₂, while hypercapnic can rise to 10% CO₂. Therefore, to mimic the environment of the tumor the method utilizes an incubator designed to provide the optimal gaseous environment corresponding to that of the tumor. Thus, the incubator will be capable of maintaining an atmosphere having an oxygen content in the range of 0.1% to 10% to 0.1% and a CO₂ in the range of 1% to 10%.

After allowing for proliferation of the cells, the cells were distributed across a plurality of test wells containing from 100 μL DMEM plus 10% FBS and allowed to attach to the surface of the test wells. Typically, the time for attachment will require about 12 hours to about 18 hours. Following attachment, the cells were treated with either a solvent control or the N-benzyl sulfonamide compound of interest dissolved in a suitable solvent such as but not limited to DMSO. Typically, about 18 hours to about 36 hours are required to determine the effect of the N-benzyl sulfonamide compound of interest on cell viability. Following treatment of the cells with the N-benzyl sulfonamide or control, the toxicity of the compound to the cells will be determined by addition of a luminescing agent. For example, 10 μl of CellTiter-Blue reagent, i.e. resazurin may be added. Typically, the resazurin is added between about 18 hours to about 36 hours after treating the cells with the N-benzyl sulfonamides or the control. The cells are allowed to consume and convert the resazurin to resorufin for about one to four hours. Subsequently, the fluorescence of resazurin is measured by excitation at 560 nm and recording the emission at 590 nm within an instrument configured to measure fluorescence intensity. Two commercially available systems are the BioTek Cytation 5 plate reader and the Promega Glomax Multi+detection system. For compounds exhibiting cytotoxicity, the half maximal inhibitory concentration (IC₅₀) values were determined using non-linear regression analysis in Graph-Prism software. The method for determining IC₅₀ values is well known in the art and will not be discussed further. As known to those skilled in the art, IC₅₀ is a quantitative measure that indicates how much of a particular inhibitory substance (e.g. drug) is needed to inhibit, in vitro, a given biological process or biological component by 50%. Therefore, each N-benzyl sulfonamide will be tested over a range of concentrations. Typically, the concentration ranges will be: 6.25 μM, 12.5 μM, 25 μM, 50 μM, and 100 μM. The control in this method is normally dimethyl sulfonamide (DMSO) or other solvent suitable for dissolving the compounds to be tested. FIG. 2 depicts the % cell viability for the indicated compounds depicted in FIGS. 1A-1L.

Method for Determining ATP Levels Following Treatment with Two Component Compositions

The conventional cytotoxicity screening method described above can identify compounds that on their own reduce cell viability; however, the above method will miss biologically active compounds that are targeting redundant pathways, pathways that are not being utilized, or pathways the cancer cells can bypass. The above method does not provide any information about a compound's biological targets or mechanism of action. Therefore, one aspect of the present invention includes a screening method suitable for identifying compounds which directly inhibit ATP metabolism by select pathways. Additionally, the following method permits identification of the pathway inhibited. Further, the rapid testing method does not require cell death during the exposure of the cells to the compound of interest.

The improved method for identifying such compounds measures ATP levels as a function of light emitted by any ATP dependent luciferase or modified luciferases, i.e. luciferase derivatives. The method uses a conventional luminescent assay to determine the number of viable cells in a culture. The improvement provided by the present method results from pre-treating cells used in the assays with a metabolic inhibitor prior to treatment with the compound of interest, or switching the cells to a media formulated to force use of a specific metabolic pathway. Luminescent assays for determining cytotoxicity are well known in the art. Assays, kits and methods for measuring ATP levels are disclosed by U.S. Pat. Nos. 7,741,067 and 7,083,911, incorporated herein by reference. Commercially available assays, marketed as the CellTiter-Glo® and CellTiter-Blue® from Promega Corporation, are particularly suited for carrying out the method described below; however, other similar luminescent or fluorescent assays will perform equally well in the described method.

The commercially available assays are configured for the purposes of determining cell viability. In normal usage, the test determines ATP levels in untreated cells, i.e. the control. Corresponding cells are treated with an agent of interest that is suspected of reducing cell viability. In the common practice, resulting data is presented as a comparison of the ATP levels in the treated and untreated cells with the decrease in ATP levels indicative of the effectiveness of the agent. The data may be presented as a direct comparison of the assay output levels or as a percentage using the untreated control cells ATP level as 100%.

In most instances, following the treatment period, the commercially available assays use a lysis buffer to break the cells apart and release ATP. The releasing agent also contains an enzyme which catalyzes a light emitting reaction. Typically, the enzyme is luciferase and its substrate D-luciferin. In the presence of ATP, luciferase catalyzes a reaction that emits light (see FIG. 7). The resulting light emission corresponds to the ATP levels of the control and the ATP levels of the treated test cells. Thus, the reduction in light intensity emission can be used to determine the level of ATP present in the treated test cells. In most cases, the light emission is quantitated using a photoluminometer. In the conventional testing, if treating cells with a compound reduces cell viability, then their ATP levels will also be reduced (since they are dead they can't make more ATP) relative to the untreated cells. Thus, the traditional CellTiter-Glo assay, commercially available from Promega, method can be described as an indirect measure of cell viability; however, what it really measures is ATP levels, which under certain conditions are indicative of cell viability. These conditions typically involve treating cells for 12-72 hours with compounds of interest, then analyzing using the reagent to produce the light emission.

In the present method, the method of using the available assays has been modified to measure the short-term effect of compounds of interest on ATP levels in the cells. The present method does not result in cell death and does not measure cell viability. In the modified method, the control and test cells are initially treated with the metabolic inhibitor for a period of about thirty minutes to about four hours. Typically, the treatment of the cells with the metabolic inhibitor or modified media is for one hour prior to adding the compound to be screened for anti-cancer properties. However, simultaneous treatment with the metabolic inhibitor and the compound being screened should provide satisfactory results. Thus, the modified luminescent assays have been adapted to screen for compounds that directly inhibit ATP metabolism. ATP synthesis in cells occurs over multiple biochemical pathways. These pathways are very responsive to metabolic inhibitors and/or changes in available nutrients. By incorporating a metabolic inhibitor or specially formulated media which is known to impact certain pathways, the disclosed method provides for measurement of a compound's direct effects on the remaining metabolic pathways available for ATP synthesis in the cell.

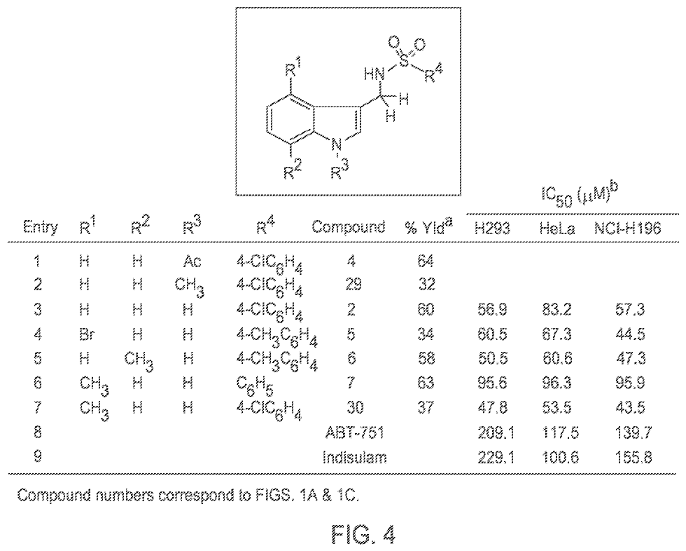

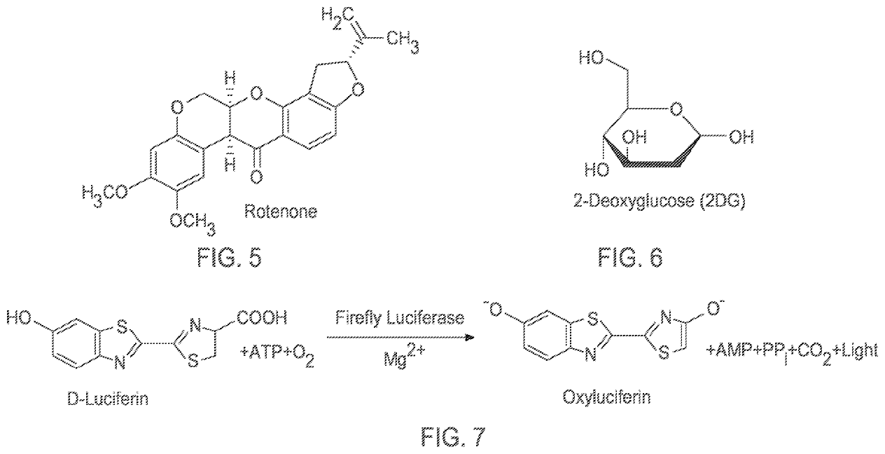

Because cancers exhibit dysregulation of metabolic pathways, compounds identified in the disclosed method are potential anti-cancer therapeutics. In this screening methodology, cells used in the assays are pretreated with a metabolic inhibitor such as 2-deoxyglucose (2-DG) (inhibits ATP production via glycolysis) or rotenone (inhibits ATP production by mitochondria). As a consequence of the pre-treatment with a metabolic inhibitor, the cell must utilize the remaining uninhibited pathways to maintain cellular ATP levels. FIGS. 5 and 6 provide the structures of rotenone and 2-DG.

Following treatment of the control and test cells with the metabolic inhibitor (or switching to a new media), the method calls for addition of the compound to be screened for anti-cancer properties to the cells. Following addition of the compound of interest, the assay is allowed to continue for about one hour to about four hours or depending on the luminescing agent up to eighteen hours. However, approximately 60 minutes will be sufficient to determine the ATP inhibiting effect of the compound to be screened on the cells. Following completion of the selected time period, the ATP level within the cell is determined. ATP levels can be determined by luminescence according to standard measuring procedures, i.e. the luminescence level of the assay from the living cells treated with the two component composition is compared to the luminescence level of the assay from the living cells without the two component composition, i.e., the control experiment. Thus, the ATP levels are determined without killing the cells during the incubation of the cells in the presence of the compound of interest. As a result, the measured reduction in ATP level corresponds directly to the inhibition of the metabolic pathways uninhibited by the metabolic blocker.

Thus, the method provides the ability to screen compounds for the ability to specifically target the uninhibited pathway being used to generate ATP. Furthermore, because cells pre-treated with a metabolic inhibitor targeting a first known pathway are forced to use an alternative known pathway that is not inhibited to maintain ATP levels, the screening method provides immediate mechanistic information about the active compound mechanism of action. These results are provided in a relatively short time period of about ninety minutes to about five hours.

A wide variety of ATP luminescing detection reagents are available commercially. So long as the reagent produces a luminescence in the presence of ATP, the reagent will be suitable for use in the present method. Suitable reagents include but are not limited to any ATP dependent luciferase such as but not limited to firefly luciferase, other modified luciferase based reagents, i.e. luciferase derivatives. According to the rapid method for determining the anti-cancer activity of a compound, a sample of living cells is distributed across a number of testing wells. Typically, 96-well plates are used; however, the number of wells is not critical to the current method. When using a 96-well plate the number of living cells will commonly be about 20,000. However, evidence presented herein shows that as few as 200 cells can be accurately evaluated. The sample wells contain a cell growth medium to promote cell health and growth and an additive to prevent bacterial contamination of the wells. One common example of the cell growth medium is DMEM with 10% FBS as described above. One example of the additive to prevent bacterial contamination is a solution of penicillin G and streptomycin referred to commonly as Pen-Strep. The Pen-Strep solution typically contains 5000 units of penicillin G and 5000 micrograms of streptomycin. Thus, the rapid determination of anti-cancer compounds can be carried out using the following method.
The desired number of cells are distributed in a 96-well plate containing 100 μL DMEM plus 10% FBS with optional Pen-Strep.
After 24 hours, cells are treated with the compound of interest or a 5% solution of dimethyl sulfoxide (DMSO) in water as a control.
The time period for exposure to the compound of interest and the DMSO control will vary depending on the luminescing detection reagent. However, when using a commercially available reagent such as resazurin (CellTiter Blue commercially available from Promega) or luciferase or a luciferase derivative (CellTiter Glo commercially available from Promega) the time period can be readily determined with reference to literature from the commercial source. Other luminescing detection agents can also be used with minimal experimentation to determine the desired compound exposure time.
Upon completion of the time period for exposure to the compound of interest or the DMSO, 10 μL of the luminescing detection reagent is added and the cells are lysed by a detergent added along with the luminescing detection reagent.
   When the reagent is resazurin, as found in CellTiter-Blue the time period for exposure to the compound of interest and the DMSO will be about 24 hours.
   When the reagent is luciferase or a luciferase derivative as found in CellTiter-Glo the time period for exposure to the compound of interest and the DMSO will be about 30 minutes to four hours.
The luminescing detection reagent is added over a period of time.
   When using resazurin, the time period for the addition of the 10 μl volume takes place over a period of about one to four hours.
   When using luciferase or a luciferase derivative, the time period is about 3 minutes to about 7 minutes, typically about 5 minutes.
During the time period of addition of the luminescing detection reagent the resulting luminescence is measured using conventional methods and devices. Suitable devices for measuring luminescence include but are not limited to a luminometer, a luminescence microplate reader or other devices with a photomultiplier tube.
The impact of the compound of interest on the cell is determined by a reduction in luminescence. If the compound of interest deactivates the cell, then the cell produces less or no ATP. As a result, the cells in the well treated with the compound of interest will have a lower luminescence value as compared to the cells in wells treated with DMSO.
The value for the cells treated with the compound of interest is reported as a “percent of control” (POC) value. The determination of POC is calculated by dividing the averaged response from duplicate wells containing the cells as treated with the compound of interest by the average response of duplicate control wells which contain only cells and DMSO (in other words, a blank control experiment).

Table 1 reports the POC values for a variety of compounds of interest. The number in the far left column of Table 1 corresponds to the compound number of compounds depicted in FIGS. 1A-1C. Each compound was tested according to the above described method. Additionally, each compound was tested in combination with a metabolic inhibitor. When using the metabolic inhibitor, the metabolic inhibitor may be added prior to the compound of interest or simultaneously with the compound of interest. To provide the best results when seeking to determine the metabolic pathway impacted by the compound of interest, the metabolic inhibitor should be added for a period of about thirty minutes to about four hours prior to the addition of the compound of interest.

As reported in Table 1 below, one group of assays included only the compound of interest. Another group of assays included the compound of interest in combination with 2-deoxyglucose and a third group of assays included the compound of interest with rotenone. Other metabolic inhibitors suitable for use in the disclosed method include but are not limited to: 2-deoxyglucose, rotenone, Lonidamine, 3-bromopyruvate, imatinib, oxythiamine, and 6-aminonicotinamide Glutaminase Inhibitor 968, 6-Diazo-5-oxo-L-norleucine, Amytal, Antimycin A, Sodium Azide, Cyanides, oligomycin, FCCP, Phloretin, Quercetin, 3BP, 3PO, DCA, NHI-1 and Oxamic acid, Fisetin, myricetin, apigenin, genistein, cyanidin, daidzein, hesperetin, naringenin, and catechin.
For assays that included 2-deoxyglucose (2-DG), a 1M aqueous stock solution was prepared. In carrying out the analysis, 1-2 μL of the 1M 2-DG was added directly to the well containing 100 μL of cells, DMEM and 10% FBS. The resulting dilution of the 2-DG provides a concentration of 2-DG at about 10-20 mM in the well. The compound of interest is added as a 100 μM solution to the well.
For assays that include rotenone, a 30 mM stock solution of rotenone in DMSO is prepared and diluted with water to provide a final 125 μM concentration of rotenone. In carrying out the analysis, 1 μL of this stock rotenone solution is added to the well containing 100 μL of cells, DMEM and 10% FBS. The resulting dilution of the rotenone stock solution provides a rotenone concentration of approximately 1.25 μM in the well. The compound of interest is added as a 100 μM solution to the well.

In Table 1 below, the cell lines tested are reported across the top row of the table. The POC values are reported for each compound of interest and each combination of interest in combination with 2DG or rotenone. A POC value of less than 50 reflects the likely inhibition of the cell line by the compound of interest or the combination of the compound of interest with the indicated metabolic inhibitor. Compound 2 in particular demonstrated reduction in luminescence, corresponding to reduced ATP activity by the cells, across many of the cell lines. When combined with 2-DG compound, 2 showed effectiveness against each cell line and a remarkable value for BxPC3 the pancreatic cancer cell line. Compound 2 would also be expected to have effectiveness against other pancreatic cancer cell lines.

### Synergistic Composition for Treatment of Cancer

While the resulting N-benzyl sulfonamides provided by the method discussed above have shown some effectiveness in vitro against select cancer cell lines, further toxicity against cancer cells would be desired. To that end, the present disclosure also provides a two-component composition which has shown a synergistic effect against cancer cells in vitro.

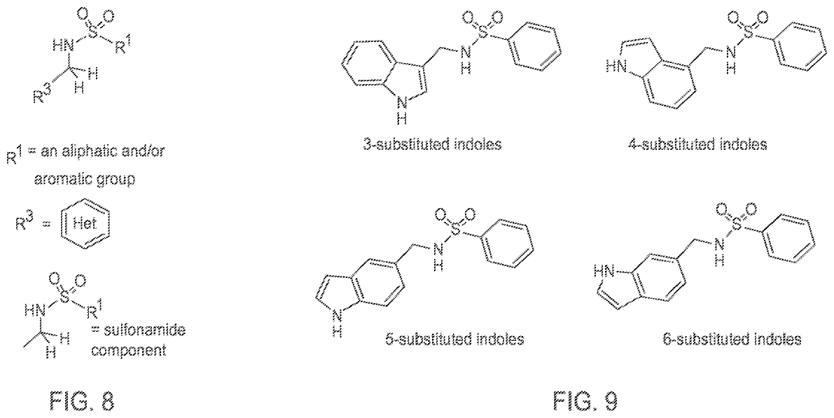

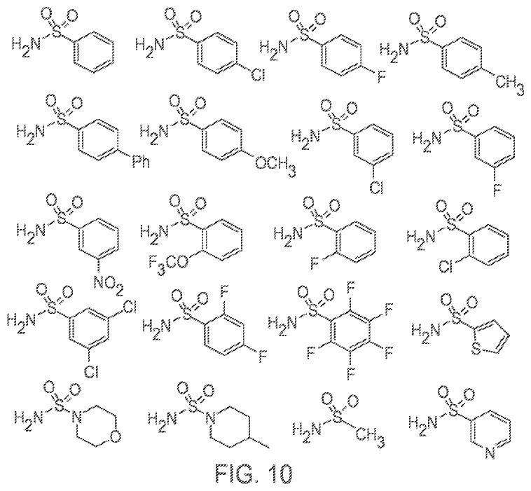

The two-component composition consists of a N-benzyl sulfonamide and a metabolic inhibitor. In one embodiment, the metabolic inhibitor is 2-deoxyglucose (2-DG). In another embodiment, the metabolic inhibitor is rotenone. Other metabolic inhibitors suitable for use in the two-component composition are: Lonidamine, 3-bromopyruvate, imatinib, oxythiamine, and 6-aminonicotinamide Glutaminase Inhibitor 968, 6-Diazo-5-oxo-L-norleucine, Amytal, Antimycin A, Sodium Azide, Cyanides, oligomycin, FCCP, Phloretin, Quercetin, 3BP, 3PO, DCA, NHI-1 and Oxamic acid, Fisetin, myricetin, apigenin, genistein, cyanidin, daidzein, hesperetin, naringenin, and catechin. While subsequent in vivo testing may determine a narrower range for the ratio of the N-benzyl sulfonamide to the metabolic inhibitor, the current ratio that has demonstrated effectiveness against cancer lines, as identified in the table below, is in the range of about 1:50 to about 1:1500. Thus, the metabolic inhibitor may comprise from about 75% by weight to about 99.99% by weight of the composition containing both N-benzyl sulfonamide to the metabolic inhibitor where the N-benzyl sulfonamide has the structure set forth in FIG. 8. Thus, the two part composition may be effective with as little as about 0.001% by weight N-benzyl sulfonamide up to about 25% by weight.

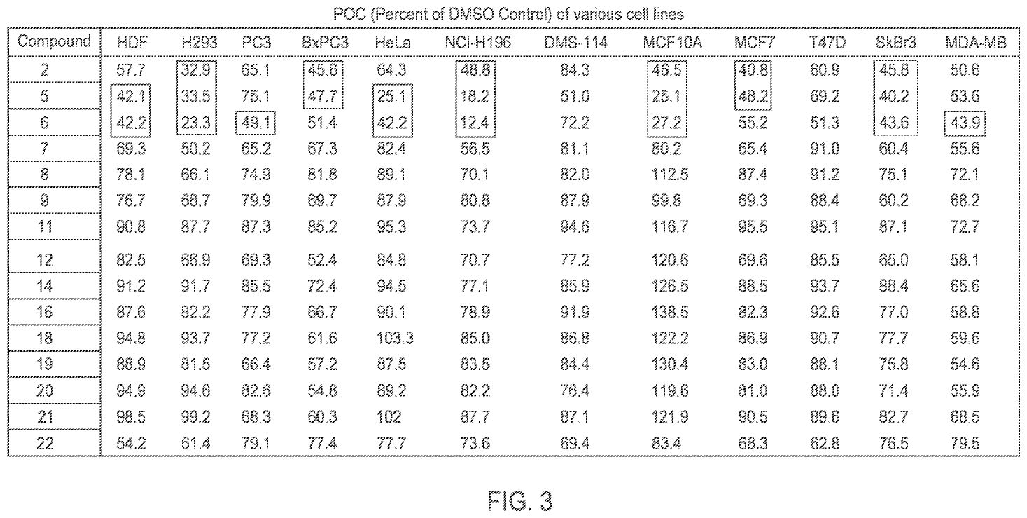

The Table of FIG. 3 provides the results of cytotoxicity testing a 100 μM concentration of compounds 2, 5-9, 11-12, 14, 16, 18-22 of FIGS. 1A and 1B against the indicated cancer cell lines using CellTiter-Blue assay with 24 hour compound incubation time. FIG. 3 reflects the percent reduction in cell viability resulting from the treatment of the indicated cancer cell lines with the indicated compounds. As indicated by the boxed values in FIG. 3, compounds 2, 5, and 6 would be considered effective against H293. Additionally, compound 5 displayed effectiveness against HeLa, NCI-H196, MCF10A. Thus, some degree of effectiveness against cancer cell lines was demonstrated. The Table of FIG. 4 provides a comparison of the IC₅₀ values of select compounds from FIGS. 1A-1L to the IC₅₀ values of two known anti-cancer sulfonamides, ABT-751 and Indisulam. As indicated by FIG. 4, the identified compounds performed remarkably better than the known anti-cancer agents. Thus, the identified compounds are expected to have greater anti-cancer potency.

Table 1 below provides the results of testing 30 different N-benzyl sulfonamides, as depicted in FIGS. 1A-1L, alone and in combination with metabolic inhibitors. The tests were carried out using the method for determining ATP levels using CellTiter-Glo reagent according to the improved method using a two hour incubation following treatment with two component compositions described in the previous section. In the following table, values less than 50% (bold and underlined) of the control value, as generated using the solvent DMSO, generally reflect effectiveness against the indicated cancer cell line. Additionally, the results reported in the table demonstrate the generally synergistic effect of the two-component composition against the tested cancer cell lines.

While the two-component composition of N-benzyl sulfonamide with rotenone showed effectiveness against several cell lines, the combination of 100 μM N-benzyl sulfonamide compound number 2 as identified in FIG. 1A with 10 Mm 2-DG demonstrated remarkable effectiveness against every cancer cell line tested. In particular, this combination showed effectiveness against the very difficult to treat pancreatic cell line. See the second row of the table below and the column identified as BxPC3. Of additional significance are compounds (such as 1, 3, 4, 22, and 23) that had little effect on the majority of cell lines, but had significant effect against the pancreatic cancer cell line BxPC3. This display of selectivity in the presence of 2-DG is significant, because it indicates compounds identified in the modified screening methodology can be combined with known drugs to specifically target and kill cancer cells. It is important to note that compounds identified using this screening methodology would have been completely missed using more traditional approaches.

TABLE 1

| All 100 uM | HDF | H293 | BxPC3 | HeLa | MCF7 | MCF10A | MDA-MB | NCI-H196 | PC3 | SkBr3 | T47D |
|---|---|---|---|---|---|---|---|---|---|---|---|
| 1 | 98.5 | 92.8 | 97.1 | 95 | 102.7 | 73.3 | 78.1 | 90.8 | 91.7 | 97.7 | 53.2 |
| 1 + 2DG | 85 | 48.2 | 27.4 | 66.3 | 86.8 | 86.7 | 62 | 68.8 | 57.3 | 72.9 | 43.2 |
| 1 + rotenone | 107.7 | 69 | 86.6 | 78.7 | 79.6 | 69 | 80.5 | 83.2 | 72.6 | 91.2 | 76.6 |
| 2 | 49.3 | 43.8 | 69 | 64.6 | 70.1 | 42.9 | 48.1 | 50.9 | 30.9 | 72.6 | 22.2 |
| 2 + 2DG | 37.5 | 20 | 13.7 | 30.8 | 25.1 | 23.7 | 37.8 | 22.9 | 16.7 | 38 | 16.6 |
| 2 + rotenone | 57.6 | 47.6 | 86.4 | 57.5 | 56.9 | 58.7 | 55.7 | 23.5 | 36.6 | 72.6 | 30.8 |
| 3 | 84.5 | 74.3 | 96.8 | 99.2 | 104.8 | 62.5 | 92.4 | 89.6 | 85 | 72.8 | 34.9 |
| 3 + 2DG | 72.7 | 42.8 | 26.1 | 67.6 | 74.5 | 72.7 | 81.8 | 55 | 59.4 | 79.4 | 37.1 |
| 3 + rotenone | 91.9 | 69.3 | 84.5 | 85.5 | 97.3 | 110.5 | 102.7 | 89.2 | 65.1 | 97.1 | 58.7 |
| 4 | 88.1 | 88.6 | 88.4 | 90.6 | 108.3 | 59.7 | 94.7 | 72.5 | 93.7 | 79.1 | 48.9 |
| 4 + 2DG | 66.8 | 60 | 21.2 | 67.4 | 82.2 | 84.8 | 63.6 | 49.6 | 61.9 | 87.5 | 36.3 |
| 4 + rotenone | 88.6 | 65.8 | 71.1 | 84.3 | 79.2 | 89.8 | 71.9 | 61.5 | 61.3 | 96.8 | 45.8 |
| 5 | 70.5 | 49.7 | 54.3 | 76.1 | 86.4 | 55.3 | 54.4 | 47.7 | 68.3 | 71.1 | 36.6 |
| 5 + 2DG | 16.1 | 17.2 | 10.5 | 22.3 | 18.7 | 15.8 | 24.7 | 4.2 | 4.8 | 37.1 | 13.8 |
| 5 + rotenone | 83.5 | 41.2 | 36.5 | 31.9 | 55.6 | 45 | 24.6 | 9.1 | 18.3 | 60 | 36 |
| 6 | 48.8 | 62.6 | 68.9 | 75.3 | 72 | 52.3 | 57.2 | 64.3 | 38.3 | 97 | 23.7 |
| 6 + 2DG | 37.7 | 31.6 | 33.4 | 47.3 | 41 | 41.3 | 58.5 | 34.3 | 35.9 | 55.9 | 19.7 |
| 6 + rotenone | 61.3 | 49.6 | 69.7 | 66.9 | 63.4 | 87.8 | 67.3 | 23.7 | 54.3 | 79.4 | 39.1 |
| 7 | 87.7 | 93.2 | 86.3 | 91 | 107.4 | 66 | 85.6 | 84.9 | 95 | 108.5 | 46.5 |
| 7 + 2DG | 60.8 | 60.9 | 60.9 | 70.6 | 91.6 | 64.5 | 81.2 | 70.1 | 59.4 | 76.2 | 40.1 |
| 7 + rotenone | 77.9 | 65.3 | 79.1 | 79.5 | 75.9 | 97.9 | 102.4 | 77.7 | 82 | 88.2 | 66 |
| 8 | 99 | 111.6 | 106.3 | 105.6 | 140.3 | 94.6 | 93.9 | 91.1 | 109.3 | 88.8 | 64.1 |
| 8 + 2DG | 91.3 | 83.5 | 87.1 | 80.8 | 86.2 | 77.9 | 91.7 | 99.2 | 87.9 | 97.1 | 94.6 |
| 8 + rotenone | 88.3 | 87.1 | 93.5 | 99.1 | 116.7 | 94.4 | 86.2 | 95.1 | 108.3 | 103.7 | 101.2 |
| 9 | 86.4 | 83.3 | 114.5 | 97.3 | 117.2 | 89.6 | 95.6 | 93.1 | 90.4 | 74.1 | 50.3 |
| 9 + 2DG | 88.5 | 76.6 | 57.6 | 80.5 | 75.8 | 88.3 | 91.7 | 86.1 | 89.3 | 89.9 | 58.9 |
| 9 + rotenone | 107.6 | 88.5 | 118.5 | 89.8 | 95.1 | 123.7 | 114 | 117.7 | 101.7 | 115.5 | 103.5 |
| 10 | 88.6 | 105.6 | 66.3 | 106.3 | 115 | 92 | 94.1 | 105.5 | 115.3 | 90.8 | 63.5 |
| 10 + 2DG | 92.7 | 84 | 62.9 | 91.3 | 121.2 | 100.4 | 86.7 | 90.8 | 97.8 | 98 | 76.6 |
| 10 + rotenone | 82.2 | 70.8 | 54.4 | 85.5 | 89.4 | 83.5 | 87.4 | 103.7 | 98.7 | 107.3 | 91.4 |
| 11 | 77.4 | 92.8 | 99.2 | 103.1 | 117.7 | 95.4 | 95.3 | 95.2 | 113.4 | 100.8 | 59.3 |
| 11 + 2DG | 101.3 | 87.4 | 83.8 | 117.6 | 98.4 | 95.8 | 82.4 | 114.7 | 94.6 | 99.7 | 90.4 |
| 11 + rotenone | 89.8 | 88.3 | 78 | 96.4 | 94.6 | 93.7 | 97.9 | 88.9 | 96.1 | 95.3 | 99.6 |
| 12 | 77.8 | 94.4 | 79.8 | 98 | 97.3 | 98.6 | 87.2 | 83.5 | 82.1 | 91.6 | 59.8 |
| 12 + 2DG | 90.9 | 85 | 57 | 78.5 | 90.8 | 88.1 | 83.9 | 117 | 89.9 | 73.5 | 58.1 |
| 12 + rotenone | 70.4 | 74.9 | 61 | 87.6 | 92.1 | 86.1 | 105.7 | 81.8 | 80.3 | 85.1 | 95.2 |
| 13 | 96.5 | 101.1 | 91.2 | 94.6 | 126.7 | 94.8 | 101.1 | 87.6 | 82.2 | 101.1 | 84.3 |
| 13 + 2DG | 104.2 | 69.4 | 74.1 | 90.7 | 89.7 | 96.1 | 81.9 | 127.4 | 97.3 | 87.2 | 88.8 |
| 13 + rotenone | 98.3 | 81.1 | 55.3 | 94.1 | 116.5 | 84 | 103.8 | 99.7 | 101.2 | 96.5 | 90 |
| 14 | 96.1 | 84.9 | 93.8 | 98.3 | 125.8 | 102.9 | 105.2 | 86.9 | 110.9 | 107.1 | 86.7 |
| 14 + 2DG | 96.3 | 82.1 | 95.4 | 106.4 | 109.8 | 119.1 | 86 | 107.3 | 101.7 | 96.9 | 106.4 |
| 14 + rotenone | 90.9 | 82.4 | 60.9 | 102.1 | 94.4 | 94.3 | 87.5 | 109 | 101.7 | 98.3 | 114.8 |
| 15 | 89.2 | 99.8 | 98.8 | 102.8 | 148 | 105.2 | 108.4 | 92.3 | 109 | 116.6 | 89.6 |
| 15 + 2DG | 100.2 | 74.7 | 70.8 | 113.9 | 103.4 | 122 | 91.2 | 115.4 | 117.6 | 123.3 | 82.5 |
| 15 + rotenone | 91.1 | 84.4 | 88.9 | 102.2 | 96.4 | 98.4 | 90.8 | 104.5 | 106.4 | 93 | 109.4 |
| 16 | 94.3 | 80.1 | 103 | 96.8 | 123.4 | 113.1 | 105.5 | 110.5 | 108.8 | 91.9 | 91.1 |
| 16 + 2DG | 95.6 | 80.7 | 95.4 | 106.8 | 93.3 | 118.9 | 104.5 | 99.2 | 113.1 | 81.4 | 111.6 |
| 16 + rotenone | 98.3 | 81.6 | 72.7 | 102.3 | 107.5 | 86.2 | 92.7 | 97.6 | 95.1 | 104 | 99.3 |
| 17 | 90.7 | 80.4 | 91.4 | 90.3 | 145.3 | 92.4 | 104.8 | 96.8 | 112.8 | 90.1 | 83 |
| 17 + 2DG | 98 | 85.8 | 101.4 | 118.8 | 114.6 | 97.1 | 89.8 | 103.8 | 93.7 | 88.8 | 89.1 |
| 17 + rotenone | 84.1 | 74.9 | 75.7 | 110.3 | 90.6 | 91.6 | 98.1 | 89.4 | 90.9 | 95.4 | 104.6 |
| 18 | 96.1 | 67.3 | 100.2 | 99.5 | 109.8 | 118.6 | 102.2 | 97.4 | 74 | 112.5 | 95.7 |
| 18 + 2DG | 93 | 96.3 | 85.4 | 100 | 137.3 | 92.2 | 86.1 | 108.3 | 94 | 107.2 | 82.8 |
| 18 + rotenone | 95.7 | 67.4 | 94.3 | 97.3 | 82 | 86.9 | 100 | 87.3 | 97.2 | 96.6 | 100.6 |
| 19 | 92.5 | 82.2 | 96.2 | 104 | 133.5 | 83.9 | 104.1 | 117.2 | 108 | 119.2 | 91.5 |
| 19 + 2DG | 98.9 | 87.2 | 98.4 | 94.8 | 104 | 95.8 | 89.5 | 107.3 | 127.1 | 105.9 | 87.4 |
| 19 + rotenone | 87.3 | 79.1 | 107.2 | 109.3 | 92.1 | 119.8 | 112 | 94.3 | 98.9 | 108.6 | 86.7 |
| 20 | 90.9 | 104.1 | 95.8 | 103.2 | 138.5 | 101.6 | 97.4 | 100.4 | 91.1 | 103.5 | 73.9 |
| 20 + 2DG | 88.1 | 84.4 | 86.4 | 91.7 | 87.3 | 78.1 | 82.9 | 90.6 | 83.8 | 96.7 | 69.7 |
| 20 + rotenone | 103.1 | 69.4 | 94.8 | 117.2 | 124.6 | 102.9 | 98.5 | 102.1 | 100.4 | 102.5 | 101.6 |
| 21 | 87.9 | 115.1 | 112.4 | 100.8 | 189.4 | 108.1 | 101 | 107.9 | 93.3 | 102 | 88.7 |
| 21 + 2DG | 97.4 | 87.8 | 106.8 | 105.8 | 121.1 | 92.8 | 100.7 | 107.4 | 131.7 | 108.5 | 99.4 |
| 21 + rotenone | 113.6 | 87 | 114.8 | 109.4 | 111 | 120 | 121.5 | 130.5 | 110.9 | 106.9 | 86.4 |
| 22 | 89.7 | 121.9 | 99.1 | 100.8 | 113.1 | 104.9 | 96.7 | 116.8 | 93 | 106 | 85.3 |
| 22 + 2DG | 76.3 | 69.9 | 42.9 | 94.5 | 107.1 | 70.2 | 86.5 | 94.6 | 94.5 | 83.5 | 62.5 |
| 22 + rotenone | 85.5 | 90.9 | 70.8 | 105.2 | 96.4 | 96.9 | 87.1 | 94.2 | 108.2 | 93.5 | 109.3 |
| 23 | 74.6 | 92.1 | 95.9 | 84.1 | 96.3 | 85 | 98.6 | 93 | 77.3 | 85.9 | 54 |
| 23 + 2DG | 90.6 | 47.4 | 34.9 | 86.9 | 93.6 | 82.8 | 85.9 | 83.5 | 92.9 | 75.5 | 46.6 |
| 23 + rotenone | 119.9 | 65.3 | 78.5 | 93.7 | 121.1 | 85.1 | 83 | 85.6 | 87.9 | 87.7 | 71.7 |
| 24 | 57.2 | 62.6 | 59 | 78.9 | 82.9 | 85.1 | 96.4 | 172 | 54.6 | 93.4 | 70.6 |
| 24 + 2DG | 65.6 | 42.1 | 43.1 | 80.4 | 55.7 | 56.8 | 41.5 | 54.6 | 52.5 | 66.5 | 49.7 |
| 24 + rotenone | 95.7 | 47.6 | 38.6 | 96.1 | 66.5 | 59.1 | 49.7 | 73.3 | 47.5 | 67.2 | 65.5 |
| 25 | 79.2 | 99.7 | 85.1 | 89.4 | 112.2 | 95.7 | 103 | 305 | 60.2 | 112.5 | 94.4 |
| 25 + 2DG | 93.6 | 79.6 | 71.5 | 90.8 | 92.9 | 98.3 | 86.2 | 92.9 | 96.8 | 88.2 | 88.9 |
| 25 + rotenone | 95.5 | 68.8 | 78.2 | 95.6 | 104.2 | 80.4 | 88.8 | 83 | 80.1 | 104.1 | 86.7 |
| 26 | 106.1 | 109.7 | 99.1 | 90.4 | 113.6 | 106 | 90.2 | 220 | 92.3 | 108.9 | 93.3 |
| 26 + 2DG | 91.1 | 72.9 | 86.6 | 106.9 | 95.9 | 99.9 | 87.6 | 91.3 | 111.5 | 95.7 | 101 |
| 26 + rotenone | 86.9 | 72 | 88.8 | 96.3 | 119.8 | 81 | 79.2 | 80.8 | 89.6 | 97.1 | 90.8 |
| 27 | 65 | 67 | 42.8 | 58.3 | 82.9 | 58.9 | 62.6 | 150 | 43.1 | 95.6 | 33.9 |
| 27 + 2DG | 38 | 30.4 | 14.2 | 104.3 | 50.2 | 27.1 | 27.3 | 117.2 | 15.2 | 62.4 | 103.1 |
| 27 + rotenone | 66.6 | 27.3 | 15.8 | 73.3 | 61.1 | 30.2 | 37.3 | 16.7 | 27.2 | 66 | 103.8 |
| 28 | 74.9 | 43.2 | 40.4 | 57.7 | 79 | 72.8 | 77.4 | 129 | 31.8 | 102.3 | 66.1 |
| 28 + 2DG | 56.1 | 49 | 41.8 | 65.6 | 60.7 | 29.5 | 45.5 | 40.4 | 56.5 | 79.6 | 33.7 |
| 28 + rotenone | 62.2 | 43.3 | 44.6 | 96.1 | 53.5 | 81.1 | 78.1 | 60.6 | 64.8 | 87.3 | 62.2 |
| 29 | 95.8 | 91.2 | 80.8 | 80.9 | 104.7 | 78.9 | 81.1 | 192 | 77.8 | 102.3 | 90 |
| 29 + 2DG | 73 | 55 | 51.7 | 83.9 | 90.9 | 46.2 | 64.7 | 51.3 | 67.5 | 72.5 | 60.7 |
| 29 + rotenone | 81.5 | 50.5 | 52.7 | 93.1 | 98 | 62.7 | 69.7 | 100.3 | 67 | 78.6 | 77.6 |
| 30 | 28.2 | 14 | 15.8 | 28.5 | 37.4 | 30 | 38.6 | 48 | 7.1 | 64.1 | 16.1 |
| 30 + 2DG | 28.4 | 12.8 | 6.3 | 17.8 | 11.3 | 8.4 | 41.5 | 18.7 | 10.6 | 41.3 | 13.2 |
| 30 + rotenone | 66.2 | 9.5 | 45.7 | 58.4 | 36 | 34.6 | 48.3 | 18.4 | 21.7 | 58.2 | 28 |

Having demonstrated the ability to identify compounds effective against cancer cells with and without the use of metabolic inhibitors, the same testing protocols may be used to determine the effectiveness of other compounds in treating cancer cells obtained from the patient and maintained in the previously described patient derived serum and/or plasma and specially formulated individualized cancer cell support media. In particular, by maintaining the cancer cells under conditions which mimic the original growth environment, one can alter the conditions in vitro thereby forcing the cancer cells to alter their metabolic pathways followed by treatment with various compounds to determine if the combination results in cell death. In other words, the changes to the metabolic pathways force the cancer cells into a condition where the remaining available metabolic pathways are potentially blocked by the compounds being screened. When a tested compound blocks cell metabolism, the cancer cells will no longer be able to produce ATP resulting in cell death. As described above, the lack of ATP can be easily identified, using the foregoing testing methods, in which case the compound that produced cell death will also be identified as a potential treatment compound for the patient's particular cancer.

Cancer cells are generally addicted to glucose (Warburg effect). Thus, screening when glucose is present will identify compounds that can inhibit glucose metabolism. The screening process outlined above utilizes glucose as an energy source for the cells. However, cells may substitute other energy sources for glucose. When doing so, the metabolic pathways will be altered. Screening of the library of compounds identified in FIGS. 1A-L with glucose as the energy source identified those compounds capable of inhibiting that metabolic pathway. To change the metabolic pathway, one can substitute a different energy source for glucose. For example, galactose is a structurally similar monosaccharide metabolized by cells via the glycolytic pathway; however, to be used in the metabolic pathway it must first be converted to glucose-6-phosphate (an early intermediate in glycolysis). The conversion galactose “costs” the cell 2 ATPs, as a result galactose metabolism proceeds through the TCA/ETC pathways in order to produce net ATP. Thus, galactose does not support the Warburg effect. Accordingly, screening of potential anti-cancer compounds when galactose is present will identify compounds that inhibit galactose metabolism or oxidative phosphorylation.

To promote the use of different metabolic pathways by the cancer cells, the cultured cells may be transferred to media formulations which differ from the individualized cancer cell support media, i.e. test media formulations. The test media formulations will differ from the individualized cancer cell support media used for cancer cell transport by addition or deletion of compounds thereby resulting in the cancer cells being forced into using different metabolic pathways. As discussed above, the change in metabolic pathways should be a condition which can be replicated in the patient and will result in the cancer cells becoming susceptible to a compound. Following screening of multiple compounds to identify those which produce cell death under those conditions, the identified compounds can be further assessed against the patient derived cancer cells to determine those compounds most likely to provide a positive outcome in the patient with minimal side effects.

As discussed above, selection of the preferred compounds for treating cancer will be based on the ability of a compound to block the metabolic pathways used by the cancer cells. Such ability may be attributable solely to the compound or a result of a determined synergistic effect resulting from the addition of a second compound or from forcing the patient derived cells into a different metabolic state by manipulating the individualized cancer cell support media supporting cell growth to provide test media formulations. The following method bypasses the current limited understanding of metabolic pathways in cancer, by allowing one to compare the outcomes of various compounds to determine efficacy. Due to the rapidity in which the following method may be carried out, the method can screen known chemotherapeutics, as well as any FDA approved drug can be evaluated for efficacy. While such an approach would likely fail to identify relevant compounds in population based studies, on an individual level it may be possible to identify and repurpose previously characterized drugs to treat a specific patient's cancer.

The use of proliferation inhibitors/apoptosis inducers requires precise dosing and of action entails inhibiting proliferation and/or inducing apoptosis, both of which can take significant time to accomplish. The mammalian cell cycle, for example, requires 18-24 hrs. to generate a new cell. Likewise, apoptotic cell death is a relatively slow, tightly controlled process that can take anywhere from 8-24 hrs. Thus, targeting/inducing these molecular events typically requires extended drug exposure because the cancer cells are not synchronized; i.e. they are in different stages of the cell cycle. This means that a significant period of time might need to elapse before a particular cancer cell becomes susceptible to the drug. A further complication is that cellular and genetic heterogeneity within the tumor means that not all cancer cells are proliferating at the same time, nor are they all susceptible to the same apoptotic inducing signals. The concept of dormancy (i.e. non-proliferating cancer cells) has emerged as a major impediment to traditional chemotherapeutic drugs because they escape death and can re-activate at a later date.

The following method preferably targets a molecular process that is common, essential, and specific to the entire tumor cell population. The disclosed method quickly disrupts metabolic pathways producing ATP and occurs independent of cell cycle position thereby producing rapid cancer cell death in a manner which decreases the probability of developing drug resistance. As described below, cancer cell metabolism represents an excellent target to accomplish these aims. Although disrupted metabolism is a hallmark of cancer, different types of cancers likely disrupt cell metabolism in a unique way. Thus, screening a wide array of FDA approved compounds for drugs that target metabolism from patient-derived cancer cells represents a unique approach for identifying effective therapeutics specific for that patient's tumor.

The following method uses the ability to measure ATP as a way to rapidly screen for metabolic inhibitors that directly decrease ATP levels. Because ATP turnover is dynamic, this assay technique only requires one to two hours to complete. In contrast, traditional cell viability assays typically require from 24 to 72 hours. Steady state ATP levels are determined by the balance between the synthesis and hydrolysis of ATP; therefore, if ATP production is inhibited then ATP levels will decrease rapidly even though cells are not yet dead. As we have shown, however, if ATP levels are lowered sufficiently and maintained at that low level, the cells eventually die (as revealed by a different cell viability assay). Thus, in the following methodology decreased cellular ATP levels resulting from a short exposure to drug are likely a direct indicator of inhibiting a metabolic pathway required for ATP production. Subsequent cell death occurs later and results from this initial rapid decrease in ATP because the cell does not have energy to perform basic functions.

As a first step, the method screens the compounds of interest to ensure that the selected compounds are not inhibiting the luciferase enzyme responsible for generating light in the presence of ATP. This step is accomplished by evaluating the effect of the target compounds on the luciferase reaction in the absence of cells with exogenously added ATP.

Subsequently, the various individual compounds and combinations of compounds will be tested against a commercially available cell line corresponding to the patient's cancer type. The commercially available cell line will be cultured in the patient's derived serum and tissue specific individualized cancer cell support media prepared as described above. This step will reduce the number of potentially useful compounds and or combinations of compounds.

Following the initial screening step, those compounds which have been identified as likely candidates for treating the patient's specific cancer will be tested against the cells derived from the patient prepared and maintained in the patient derived serum and/or plasma and individualized cancer cell support media selected for the specific organ or tissue location of the cancer. Testing conditions may include: (1) use of solely the identified compounds; (2) use of the identified compounds in combination with metabolic inhibitors; (3) either of the foregoing approaches under conditions where the individualized cancer cell support media supporting the cells has been modified to provide test media formulations designed to induce metabolic changes in the cells. When using metabolic inhibitors, the patient derived cancer cells may be exposed to the metabolic inhibitors prior to treatment with the identified compound or simultaneously with treatment by the identified compound.

The final screening steps are outlined above in detail in the section entitled “Method of in vitro Cytotoxicity Screening.” Thus, as a summary, the method of identified compounds particularly suited to treating the patient's cancer includes the following steps:
preparing patient derived cancer cells in patient derived serum and/or plasma followed by preparing the individualized cancer cell support media specially formulated to imitate the patient's body chemistry;
screening the compounds of interest to ensure that the selected compounds do not inhibit the luminescing detection agent, typically luciferase enzyme, responsible for generating light in the presence of ATP;
providing a first sample of living cells in a first test well, the first sample of cells being selected from a commercially available cell line corresponding to the patient's cancer type;
providing a second sample of living cells in a second test well, the second sample of cells being selected from a commercially available cell line corresponding to the patient's cancer type;
treating the first sample of living cells with the compound of interest;
treating the second sample of living cells with a suitable control compound;
selecting a luminescing detection agent suitable for the compounds of interest and determining the time period for exposure of the first sample of living cells to the compound of interest which must pass prior to addition of the selected luminescing detection agent to the first sample of living cells and the second sample of living cells based on the selected luminescing detection agent;
adding the luminescing detection agent to the first sample of living cells over a period of time as determined by the selected luminescing detection agent and adding the luminescing detection agent to the second sample of living cells over a period of time as determined by the selected luminescing detection agent;
measuring the resulting luminescence produced by the first sample of living cells;
measuring the resulting luminescence produced by the second sample of living cells;
the difference between the luminescence of the first sample of living cells and the luminescence of the second sample of living cells reflects a reduction in ATP levels in the first sample of living cells which is indicative of the cytotoxicity of the compound of interest;
the step of treating the first sample of living cells with the compound of interest does not result in cell death; and,
determining if the selected compound will likely result in cell death in the cancer cells.

The foregoing steps are repeated until a desired number of compounds have been identified for testing against the patient derived cells. The foregoing steps are then repeated using the patient derived cells in patient derived serum and/or plasma and the subsequently prepared individualized cancer cell support media selected to support the patient derived cells. As previously noted, the steps of adding the compound of interest may be preceded by any one of the following additional steps or combination thereof:
treating the patient derived cells with a metabolic inhibitor followed by treatment with the compound of interest;
modification of the individualized cancer cell support media selected to support the patient derived cells in a manner to provide test media formulations designed to change metabolic processes in the patient derived cells;
treating the patient derived cells simultaneously with a metabolic inhibitor and the compound of interest;
treating the patient derived cells with two or more compounds of interest simultaneously or in sequence.
   Thus, the method of screening compounds for treating the patient's specific cancer cells is carried out in vitro but provides an environment which closely mirrors the in vivo conditions of the cancer in the patient. Further, this method may be carried out by proceeding directly to the second step and testing a larger group of compounds to identify the preferred treatment compounds. Thus, the first step is an optional step. Without intending to be bound by theory, we believe that many cancer cells display decreased p27kipl and that activation of this “switch” can be triggered by the targeted compounds thereby leading to reduced ATP production and cell death.

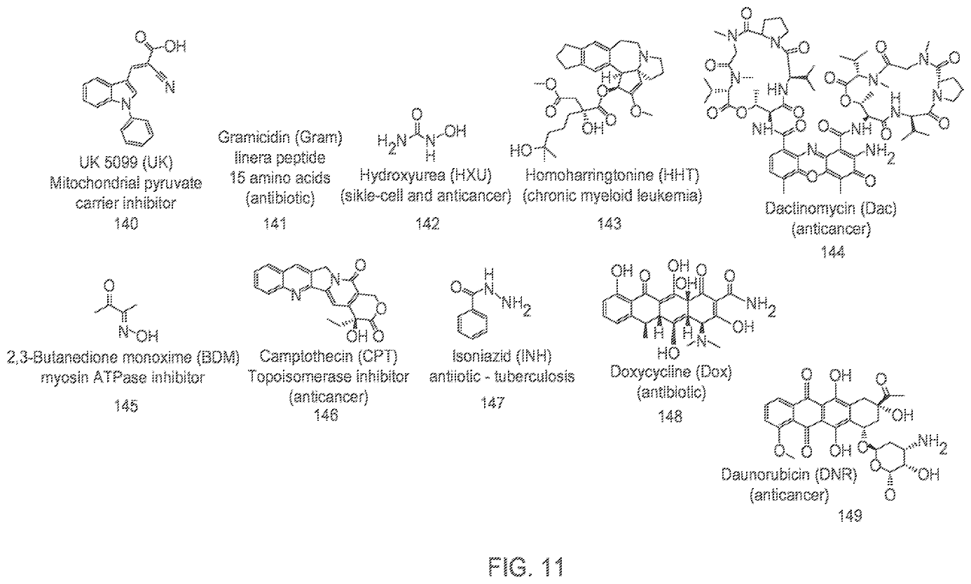

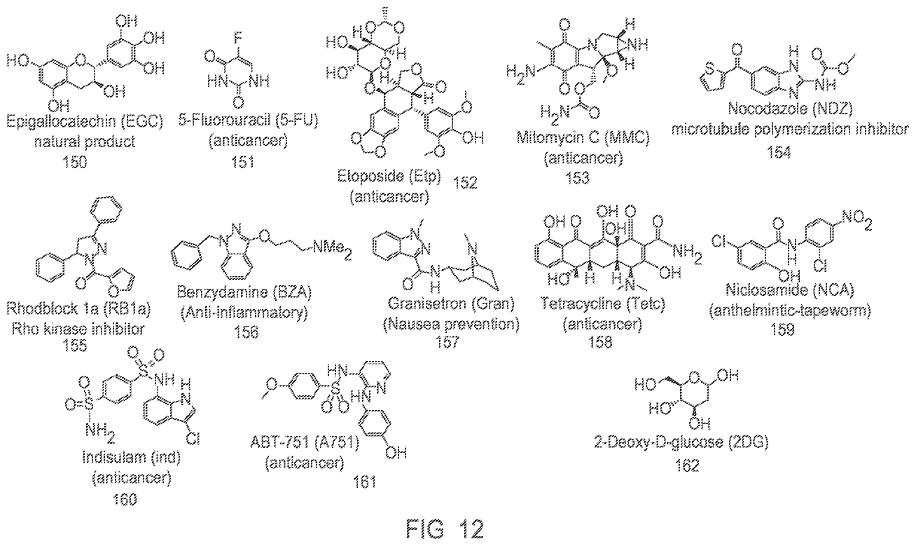

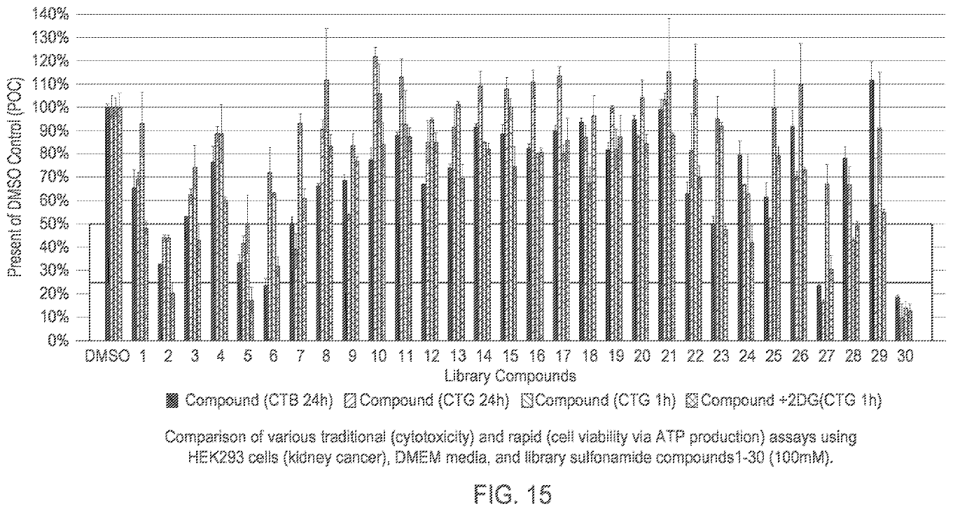

The following test results demonstrate the effectiveness of the above describe screening method. The following tests results were obtaining using the above described screening method on different types of commercially available cancer cell lines as a demonstration of the ability to rapidly test and confirm the effectiveness of compounds of interest. When used to identify compounds for specific patients, the following steps will be carried out:
Individualized patient cells will be acquired from a tumor biopsy and cultured/grown in patient blood serum and/or plasma. (In most instances the cells will be obtained using a needle biopsy; i.e. fine needle aspiration biopsy. Optionally, the cells may be obtained from resected tumor (i.e. excisional biopsy, which is an attempt at complete tumor removal), and/or a core biopsy; (i.e. removal of part of the tumor). Alternatively, cells may be obtained from blood draw samples, which would contain leukemia, lymphoma and myeloma cells.)
The cells (as few as 100 cells per experiment) will be screened directly in patient blood serum and/or plasma in the absence or presence of glycolysis inhibitor, 2-deoxyglucose (2DG) using a rapid (1-2 hr) screening assay (CellTiter-Glo, or “CTG”).
   NOTE: while the discussion herein focuses on CTG, other assays suitable for measuring changes in ATP levels will perform equally as well. The primary criteria in the disclosed method being the ability to measure ATP levels against a control to determine cell response to the compounds of interest.
The screening method described above requires only one to two hours to provide results comparable to the current standard test methods which require 24 to 72 hours. The ability of the screening method to provide these results is demonstrated by FIG. 15.
   For the library of compounds identified in FIGS. 1A-C, i.e. compounds 1-30, the graph of FIG. 15 demonstrates screening results on HEK293 (kidney cancer) cells using a traditional cytotoxicity screening (CellTiter-Blue, 24 hr; CellTiter-Glo, 24 hr), a rapid screening for ATP production (CellTiter-Glo, 1 hr), and a rapid ATP production with metabolic inhibitor, 2DG (CellTiter-Glo, 1 hr, +2DG). Results indicate that comparable results to traditional methods can be obtained using the rapid assays. In some cases, enhanced sensitivity is also observed (compound 5). Additionally, FIGS. 2-7 demonstrate the utility of 2DG as a metabolic inhibitor when used in combination with compounds 1-30.
The patient cells will be incubated in the individualized cancer cell support media, i.e. the patient's cancer cells are incubated in patient derived serum and/or plasma which has been further modified by addition of compounds necessary to mimic the organ from which the cancer cells were obtained.
   For the tests below (demonstration purposes), the cancers cells in media which corresponded to patient derived serum and/or plasma were transferred to a commercially available cell growth media such as DMEM or L15 in order to conduct the rapid screening assay. DMEM contains glucose, but L15 has substituted glucose with galactose. This means that in DMEM, 2DG is added to inhibit glycolysis; but in L15, the addition of 2DG is unnecessary to achieve the same result as the lack of glucose in L15 precludes access to the metabolic pathway that would utilize glucose. If a compound is potent by itself in L15, it can be thought of as a compound that would be potent when combined with 2DG in DMEM media.
The test results below demonstrate the ability to rapidly screen FDA approved drugs and other compounds for potency in the treatment of a variety of mammalian cancerous and non-cancerous cell lines and subtypes alone or in combination with 2-deoxyglucose (2DG). The compounds identified with compound numbers 140-161 as depicted in FIGS. 11 and 12 were tested in this manner. NOTE: compound number 141 is Gramicidin, also known as Gramicidin D. This antibiotic is identified by CAS number 1393-88-0.
The rapid screening assay provides evidence of unexpected drug activity for known drugs against cancer cell lines. Thus, the screening of FDA-approved drugs for synergistic activity with metabolic inhibitors can provide anticancer drugs from drugs currently used for other purposes. In addition, this shows that screening anticancer drugs against cancer cells can rapidly identify which drug is truly best suited for the individual cell source.

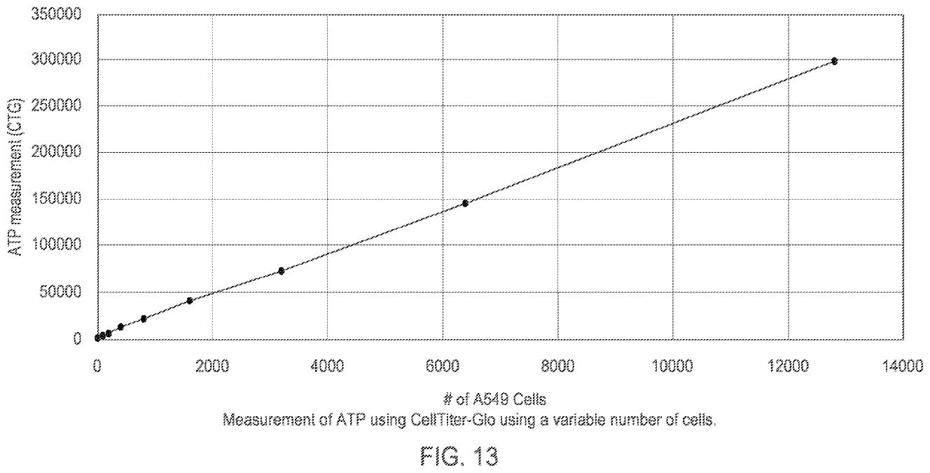

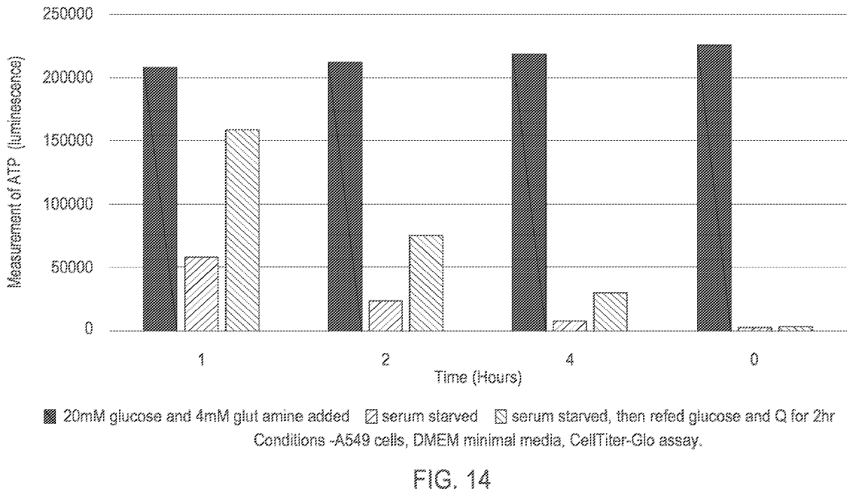

FIG. 13 demonstrates that ATP levels can accurately be measured using as few as 100 cells. Thus, use of cells obtained from biopsy procedures will provide a sufficient source of cells for practicing the above described screening method. FIG. 14 demonstrates that depleting ATP levels rapidly leads to a failure of cells to recover. In other words, the graph of FIG. 14 shows that by 4 hrs, re-feeding the cells does not restore ATP levels (darker grey bar). If the cells can't restore ATP they will die, so this supports our claim that short term measurement of metabolic inhibition induces and is indicative of future cell death. The data depicted in FIG. 14 was generated under the following conditions: A549 cells, DMEM minimal media, CellTiter-Glo assay. “Serum starved”=DMEM minimal media without added glucose and amino acids. For the “serum starved, then refed” cells, nutrients (20 mM glucose and amino acids) are added at the indicated time @37° C.+CO₂, and CTG assay performed 2 hours after the addition of the nutrients.

FIG. 15 demonstrates the ability to perform the screening method in two hours or less with results comparable to traditional 24 to 72 hour tests. The test results depicted in FIG. 15 utilized compounds 1-30 of FIGS. 1A-1C. The graph represents screening results on HEK293 (kidney cancer) cells using a traditional cytotoxicity screening (CellTiter-Blue, 24 hr; CellTiter-Glo, 24 hr), a rapid screening for ATP production (CellTiter-Glo, 1 hr), and a rapid ATP production with metabolic inhibitor, 2DG (CellTiter-Glo, 1 hr, +2DG). As used below, CTG=CellTiter-Glo.

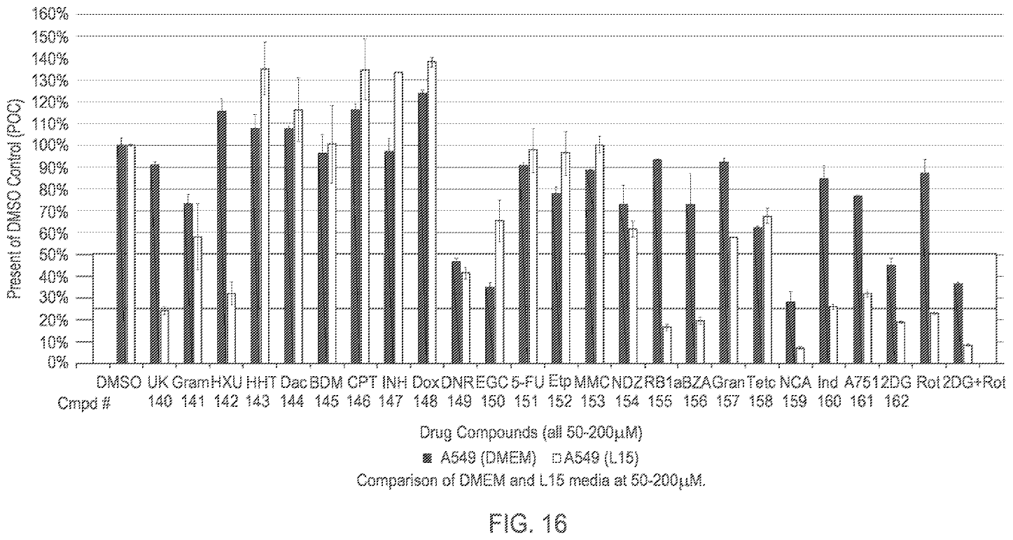

The data presented in FIGS. 16-33 was developed using compounds 140-161 as depicted in FIGS. 11 and 12. The graphs of FIGS. 16-33 provide a direct comparison of the rapid screening method wherein the tests were carried out over one hour (CTG) using Dulbecco's Modified Eagle Medium (DMEM), without 2DG and L15 media (described below), a cell media that is missing glucose; the equivalent to DMEM plus 2DG. Compounds 140-161 were screened against lung cancer cells designated A549. Similar results are obtained in most cases, but a few examples show significant enhancement of activity in L15. As used in the discussion below, Rot=rotenone, and 2DG+Rot=a positive control (ATP should be inhibited to a high degree). The term positive control indicates that the compound or combination of compounds would produce a reduction in ATP over the given period of time. Thus, compounds that produce comparable results would be considered effective against the given cancer cell line.

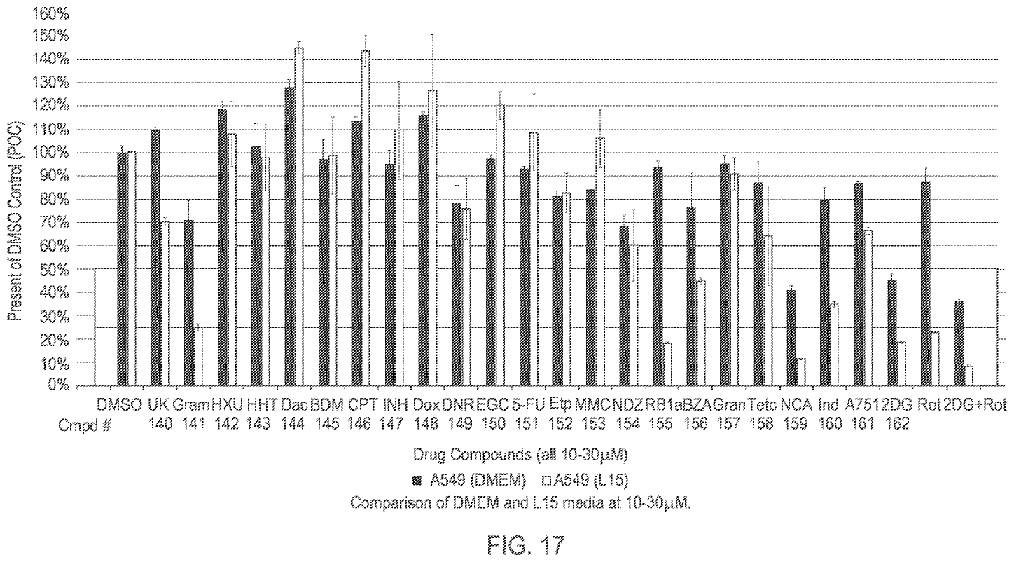

FIG. 16 provides a comparison of DMEM and L15 media at 50-200 μM. After one hour of exposure and assessment of the ATP values using CellTiter-Glo assay, the “percent of control” (POC) value is determined with respect to the DMSO control. As reflected by FIG. 16, the following compounds, at the indicated concentrations, would be considered effective against the cancer cells of line A549: 140, 142, 149, 150, 155, 156, 159, 160, 161. The same compounds were tested at 10-30 μM with the results depicted in FIG. 17. At these concentrations, the following compounds would be considered effective at least one concentration level: 141, 155, 156, 159, 160.

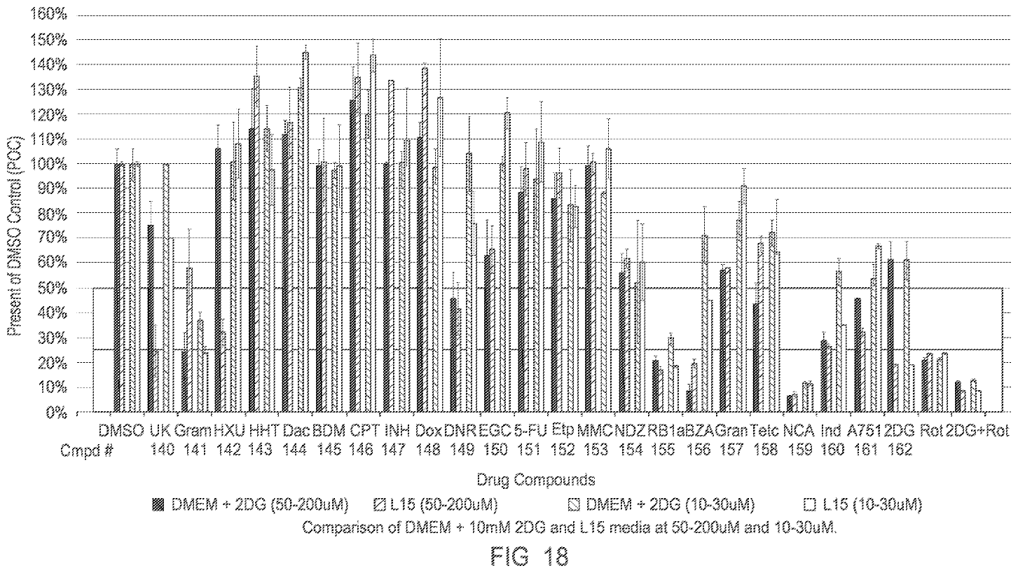

FIG. 18 provides a comparison of DMEM with 10 mM 2DG with the compounds of interest at concentrations of 50 to 200 μM and 10 to 30 μM to L15 Media with the compounds of interest at concentrations of 50 to 200 μM and 10 to 30 μM. The compounds were tested against the same cancer cell line with one hour of exposure and CTG assay with POC reported against a DMSO control. Under the reported conditions the following compounds would be considered to have effectiveness in treating the cancer cell line at least one concentration level: 140, 141, 149, 155, 156, 159, 160, 161.

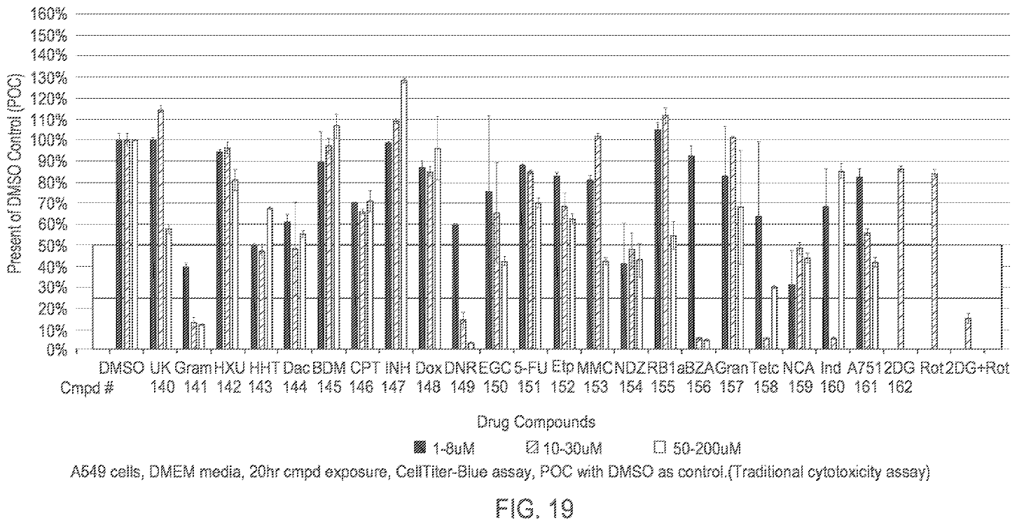

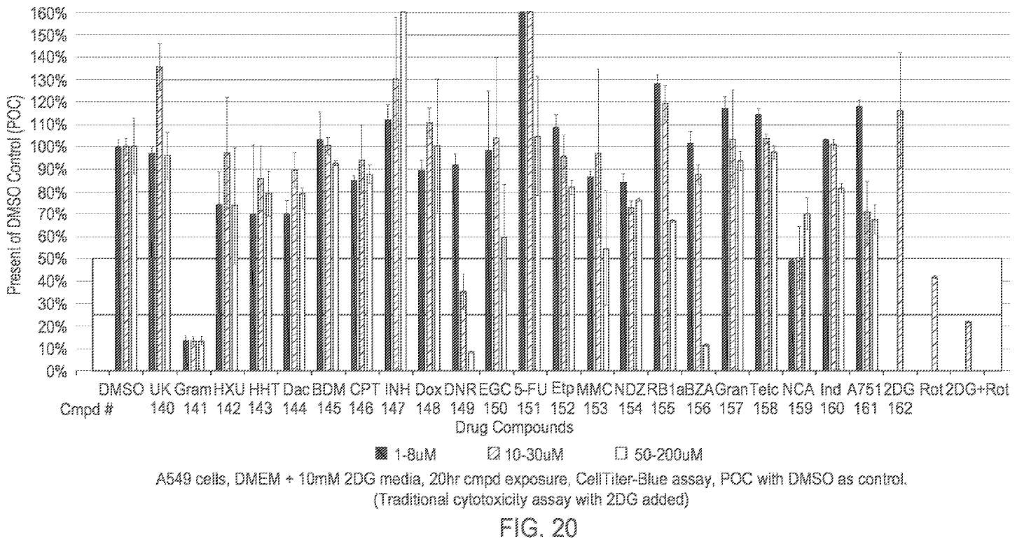

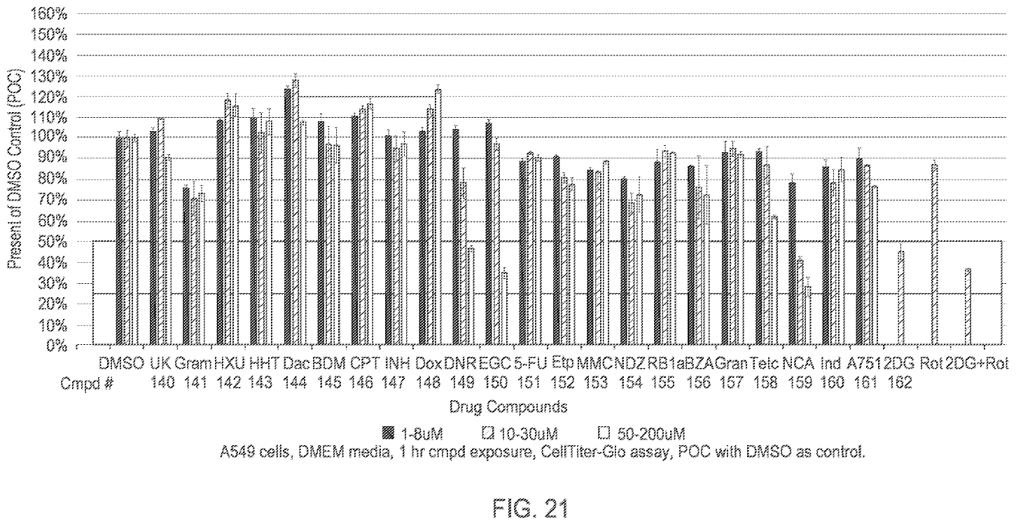

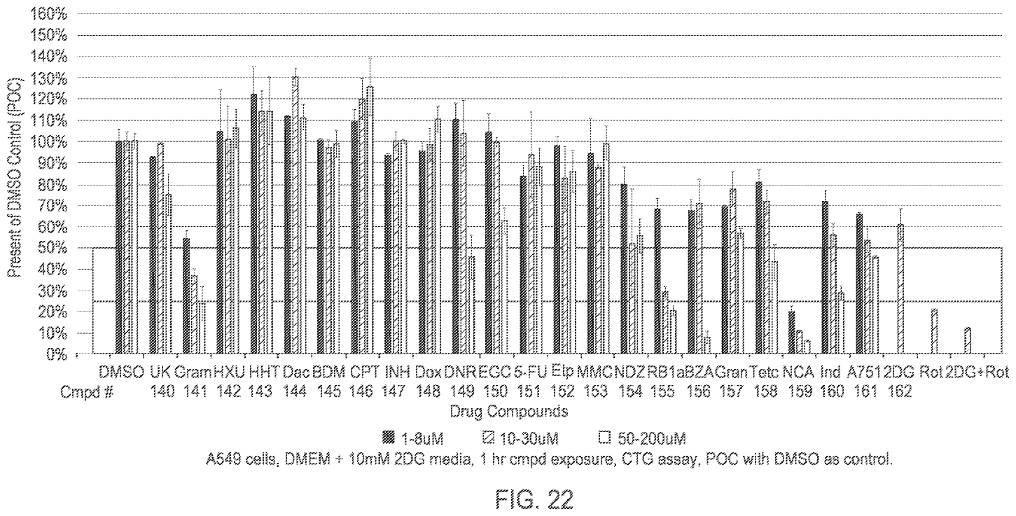

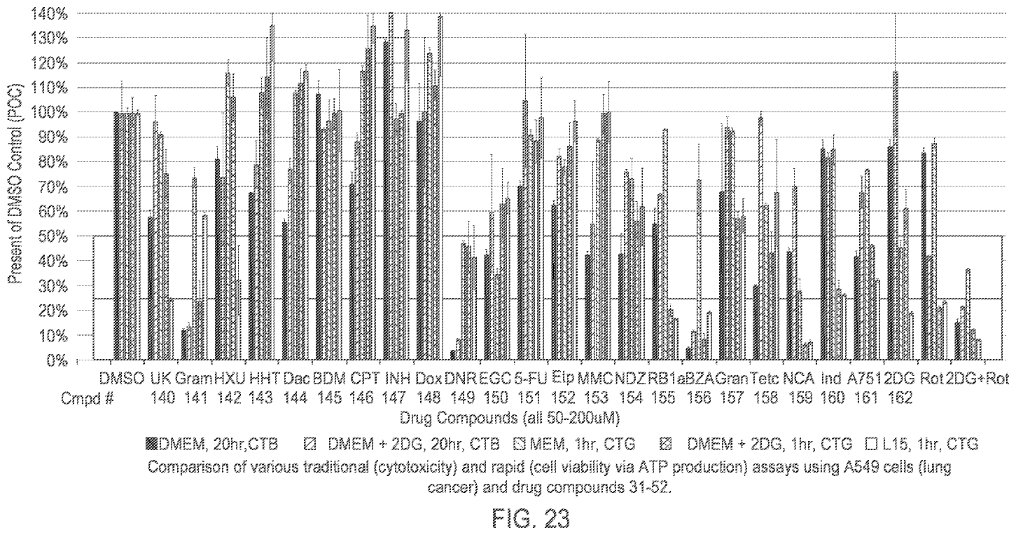

FIG. 19 provides the results of testing compounds 140-161 against lung cancer cell line A549 in DMEM media using the 20 hour method with CellTiter-Blue (CTB) assay both methods used DMSO as the control. FIG. 20 provides the results of testing compounds 140-161 against lung cancer cell line A549 in DMEM media with 10 mM 2DG using the 20 hour method with CellTiter-Blue (CTB) assay both methods used DMSO as the control. Thus, FIGS. 19-20 when compared FIGS. 21-22 demonstrate that the rapid testing method provides comparable results. FIG. 23 provides a side-by-side comparison of the one hour to 20 hour test results.

FIGS. 21 and 22 provide test data developed using the rapid testing method described above. In FIG. 21, compounds were tested for effectiveness as concentrations ranging from 1-8 μm, 10-30 μM and 50-200 μM in DMEM media for one hour using CTG assay to measure ATP levels. The POC level is reported against DMSO as the control. In FIG. 22, the DMEM media further contains 10 mM 2DG. According to FIG. 21, the following compounds would be expected be effective against the cancer cell line: 149, 159. According to FIG. 22, the following compounds would be expected to be effective, at least one concentration level, when combined with 2DG: 142, 155, 156, 158, 159, 160, 161.

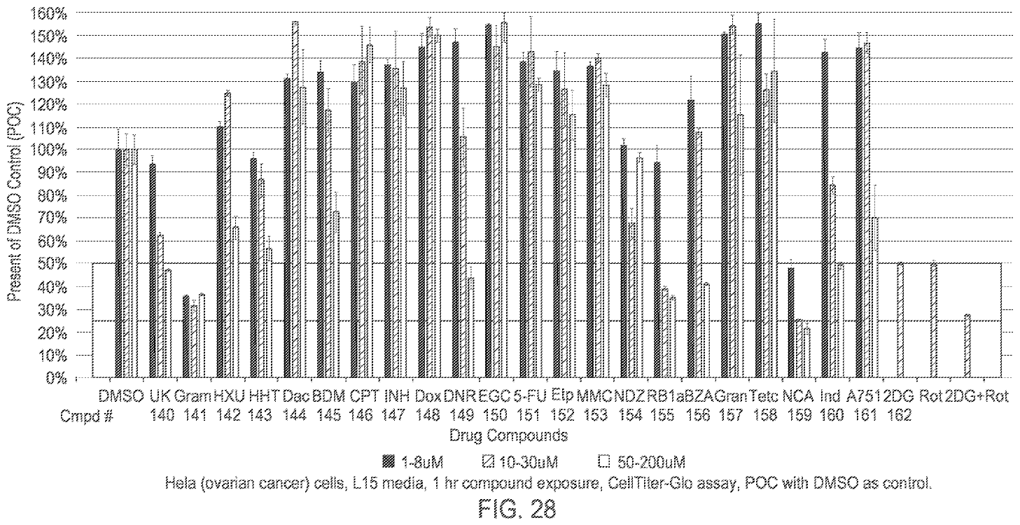

As noted above, FIG. 23 combines the data from FIGS. 19-22. Of particular interest in FIG. 23 is the unexpected effectiveness of compound number 156 (benzydamine). Benzydamine is an anti-inflammatory, not a known anticancer compound. Thus, the present method clearly provides a rapid testing method for identifying previously approved compounds that may also have anti-cancer properties. In this instance, benzydamine was tested at a concentration of 145 μM in L15. As reflected in the FIGS. 28-31, benzydamine was also effective against ovarian cancer, breast cancer and pancreatic cancer at this concentration.

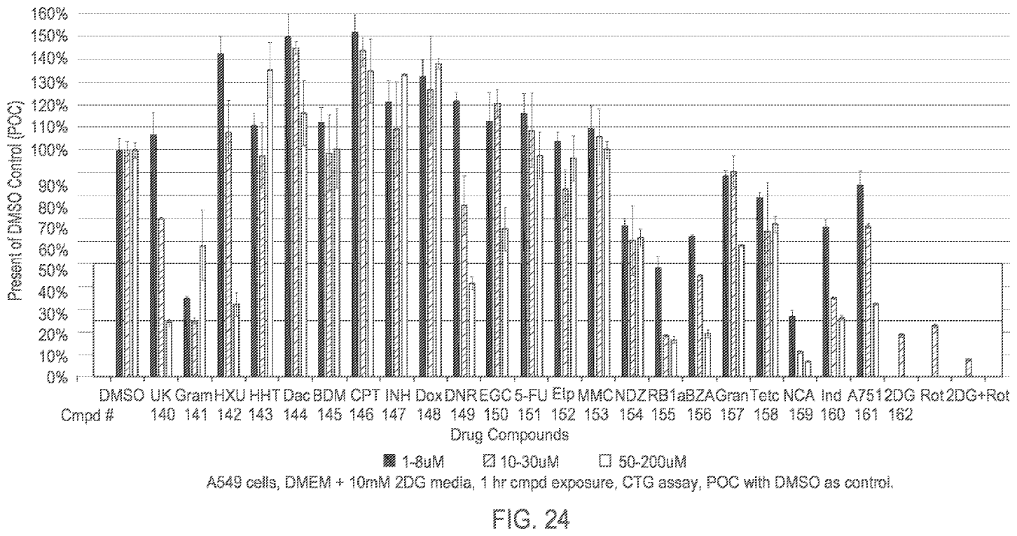

FIG. 24 provides data reflecting the testing of compounds 140-161 against lung cancer cells A549 using L15 media under the one hour testing protocol with CTG assay to determine ATP levels. POC values are based on DMSO as control. In FIG. 24, the compounds were tested at three different concentration ranges: 1-8 μm, 10-30 μM and 50-200 μM. As depicted in FIG. 24, the following compounds would be considered likely to be effective against the cancer cell line at least one concentration level: 140, 141, 142, 149, 155, 156, 159, 160, 161.

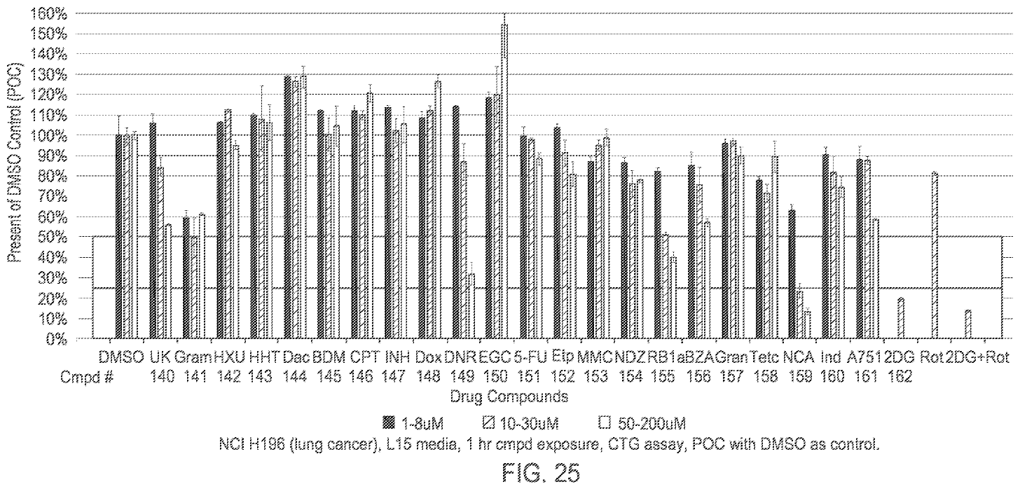

FIG. 25 provides data reflecting the testing of compounds 140-161 against lung cancer cell line designated NCI H196. The rapid testing, one hour, test used L15 media with CTG assay and POC based on DMSO as control. Compounds that would be considered likely effective for at least one concentration level include: 141, 149, 155, 159.

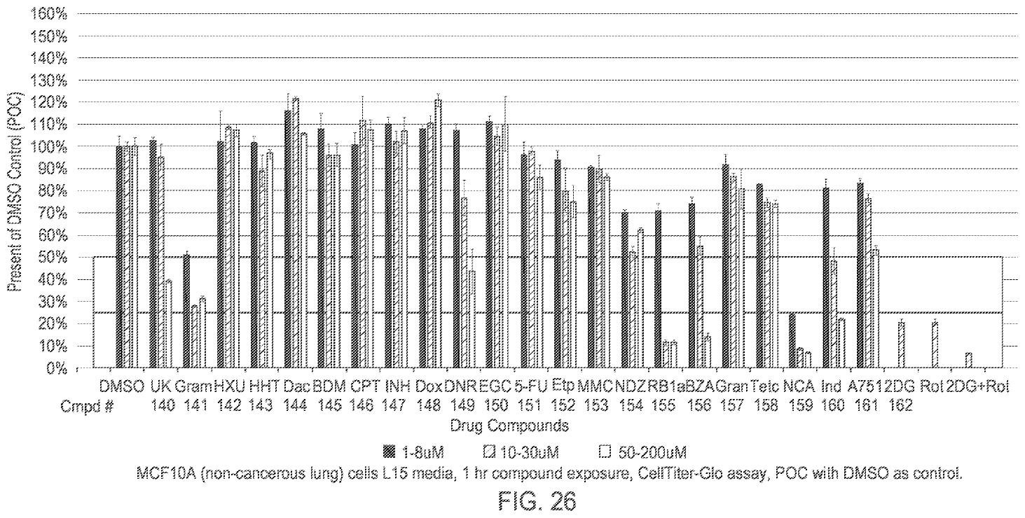

FIG. 26 provides data reflecting the testing of compounds 140-161 against non-cancerous lung cell line designated MCF10A. The rapid testing, one hour, test used L15 media with CTG assay and POC based on DMSO as control. Compounds that would be considered likely effective for at least one concentration level include: 140, 141, 149, 155, 156, 159, 160.

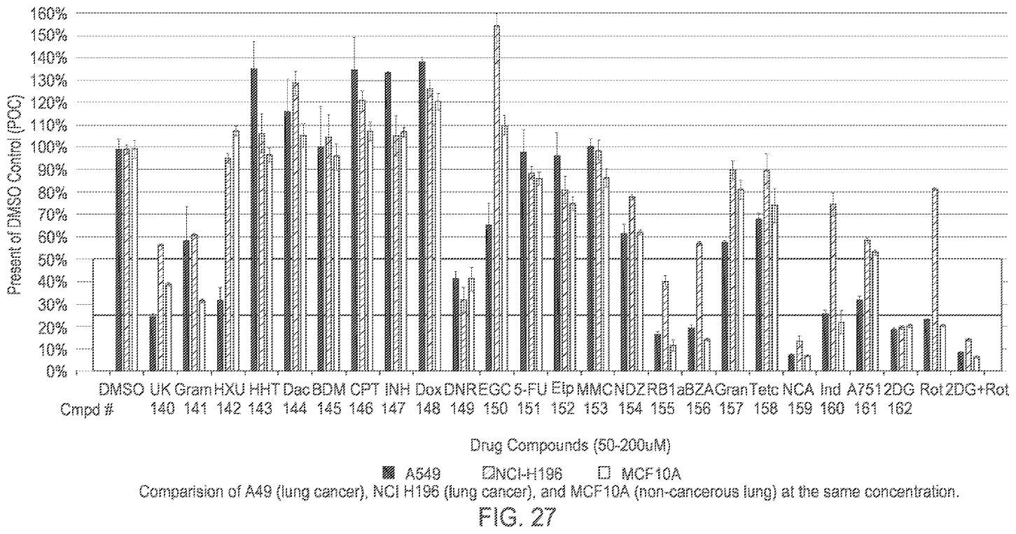

FIG. 27 provides a comparison of the effectiveness of the compounds against A549, NCI H196 and MCF10A at concentrations in the range of 50 μM to 200 μM. The data provided in FIG. 27 corresponds to that presented in FIGS. 24-26. FIG. 27 demonstrates subtype specificity for compounds: 141, 156 and 160. FIG. 27 also demonstrates specificity toward cancer over non-cancerous cells for compounds 142 and 161.

FIG. 28 provides data reflecting the testing of compounds 140-161 against ovarian cancer cell line designated HeLa. The rapid testing, one hour, test used L15 media with CTG assay and POC based on DMSO as control. Compounds that would be considered likely effective for at least one concentration level include: 140, 141, 149, 155, 156, 159, 160.

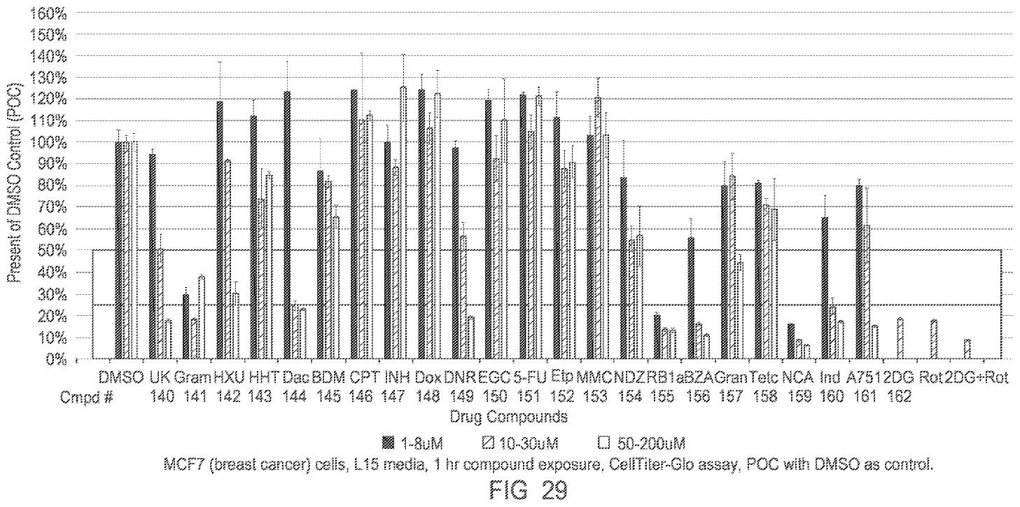

FIG. 29 provides data reflecting the testing of compounds 140-161 against breast cancer cell line designated MCF7. The rapid testing, one hour, test used L15 media with CTG assay and POC based on DMSO as control. Compounds that would be considered likely effective for at least one concentration level include: 140, 141, 142, 144, 149, 155, 156, 157, 159, 160, 161.

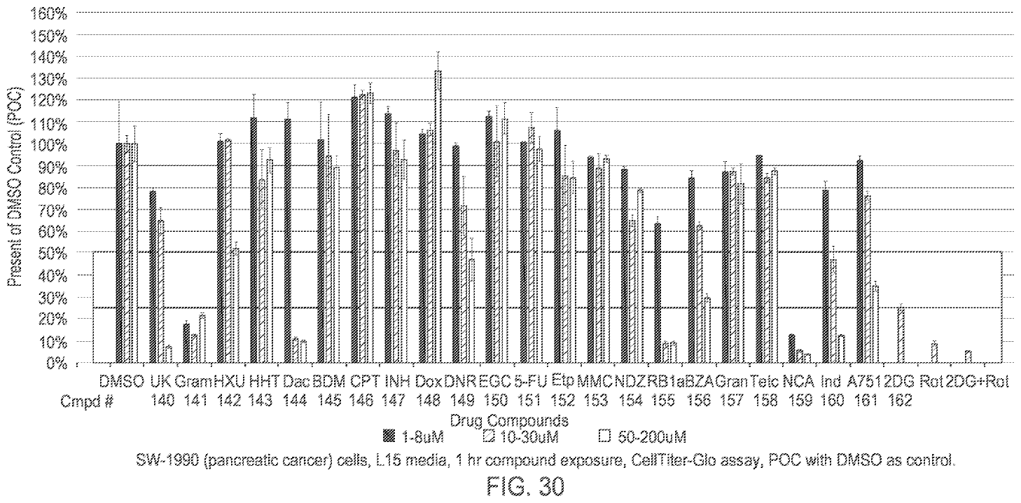

FIG. 30 provides data reflecting the testing of compounds 140-161 against pancreatic cancer cell line designated SW-1990. The rapid testing, one hour, test used LI5 media with CTG assay and POC based on DMSO as control. Compounds that would be considered likely effective for at least one concentration level include: 140, 141, 144, 149, 155, 156, 159, 160, 161.

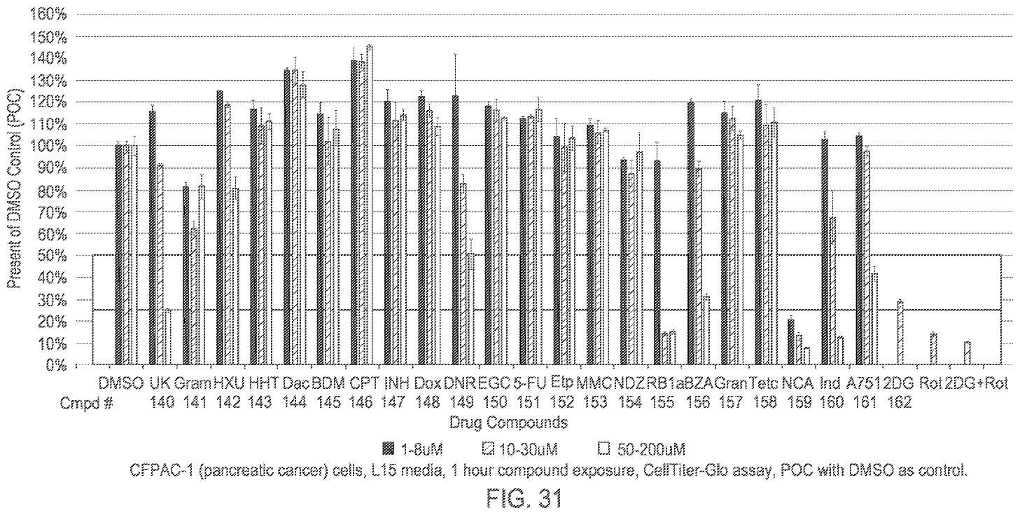

FIG. 31 provides data reflecting the testing of compounds 140-161 against pancreatic cancer cell line designated CFPAC-1. The rapid testing, one hour, test used L15 media with CTG assay and POC based on DMSO as control. Compounds that would be considered likely effective for at least one concentration level include: 140, 149, 155, 156, 159, 160, 161.

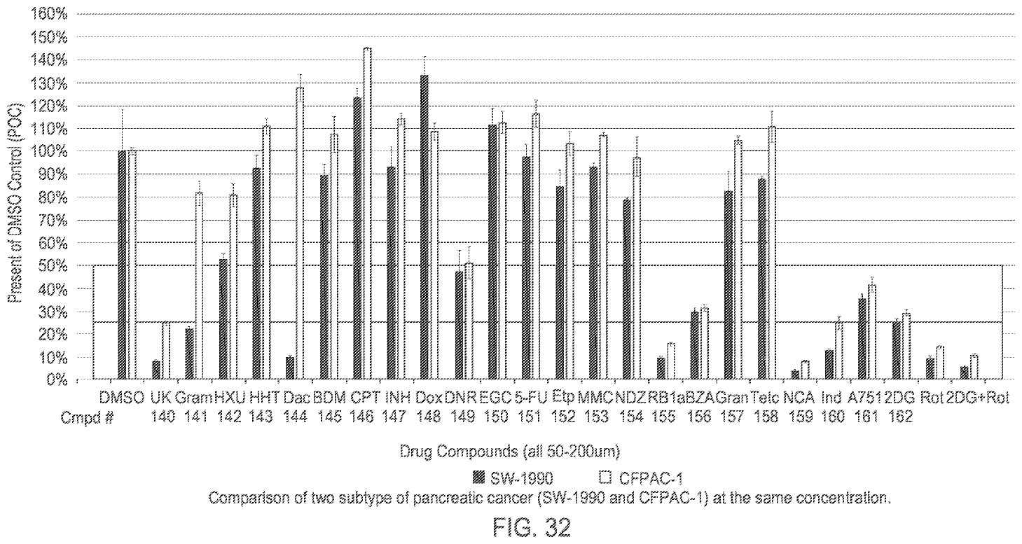

FIG. 32 compares the testing of compounds 140-161 at a concentration in the range of 50 μM to 200 μm against the two pancreatic cells lines from FIGS. 30 and 31. The test results demonstrate that different pancreatic cell lines can be impacted by differing compounds.

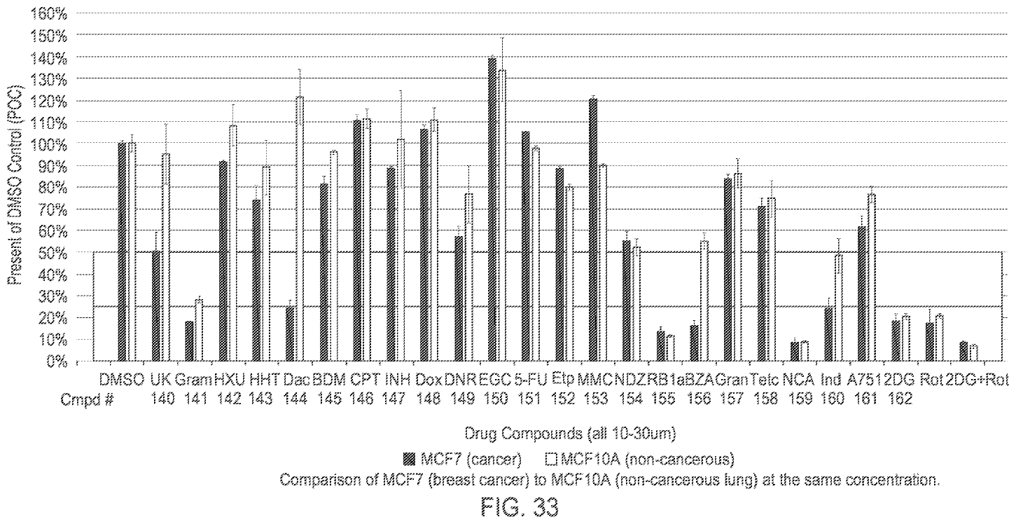

FIG. 33 compares the impact of compounds 140-161 on breast cancer cell line MCF7 and non-cancerous lung cells MCF10A. All compounds tested were in the range of 10 μM to 30 μM. As reflected by the table, compounds 144 and 156 had significantly more impact on the cancerous cells.

While L15 is a well known media, the following describes the composition of L15 used in the above described tests:
Composition of Liebovitz's L15 cell culture medium (sold by ATCC: ATCC 30-2008):
Inorganic Salts (g/liter)
   CaCl2 (anhydrous) 0.14000
   MgCl2·6H2O 0.20000
   MgSO4 (anhydrous) 0.09767
   KCl 0.40000
   KH2PO4 (anhydrous) 0.06000
   NaCl 8.00000
   Na₂HPO4 (anhydrous) 0.19000
Amino Acids (g/liter)
   L-Alanine 0.22500
   L-Arginine (free base) 0.50000
   L-Asparagine·H2O 0.25000
   L-Cysteine (free base) 0.12000
   L-Glutamine 0.30000
   Glycine 0.20000
   L-Histidine (free base) 0.25000
   L-Isoleucine 0.12500
   L-Leucine 0.12500
   L-Lysine-HCl 0.09370
   L-Methionine 0.07500
   L-Phenylalanine 0.12500
   L-Serine 0.20000
   L-Threonine 0.30000
   L-Tryptophan 0.02000
   L-Tyrosine·2Na·2H2O 0.43000
   L-Valine 0.10000
Vitamins (g/liter)
   Choline Chloride 0.00100
   Riboflavin-5-PO4·Na·2H2O 0.00010
   Folic Acid 0.00100
   myo-Inositol 0.00200
   Nicotinamide 0.00100
   D-Pantothenic Acid 0.00100
   (hemicalcium)
   Pyridoxine·HCl 0.00100
   Thiamine·PO4·Cl·2H2O 0.00100
Other (g/liter)
   D-Galactose 0.90000
   Phenol Red, Sodium Salt 0.01000
   Sodium Pyruvate 0.55000

Other embodiments of the present invention will be apparent to one skilled in the art. As such, the foregoing description merely enables and describes the general uses and methods of the present invention. Accordingly, the following claims define the true scope of the present invention.

1. A method for assessing the in vitro effectiveness of a compound against a specific cancer type comprising:
obtaining cancer cells from an organ within a patient;
identifying the organ from which the cancer cells were obtained, the organ having an in vivo environment which includes amino acids, vitamins and inorganic salts in various concentrations;
determining the amino acids, vitamins and inorganic salts and concentrations of the amino acids, vitamins and inorganic salts in the in vivo environment of the organ from which the cancer cells were obtained;
preparing individualized cancer cell support media formulated to correspond to the in vivo environment within the organ within the patient from which the cancer cells were obtained;
incubating the cancer cells in the individualized cancer cell support media under conditions which will promote cancer cell growth using metabolic pathways corresponding to cancer cell growth in the patient wherein the cancer cells in the individualized cancer cell support media are maintained at a temperature between about 35° C. and 39° C. under an atmosphere containing carbon dioxide;
treating the cancer cells with at least one compound of interest;
adding a luminescing agent to the cancer cells;
measuring a level of light emission of the luminescing agent and comparing the level of light emission to a level of light emission of a control; and,
determining an IC₅₀ value for the compound of interest thereby assessing the in vitro effectiveness of a compound against a specific cancer type.

2. The method of claim 1, wherein the individualized cancer cell support media comprises:
amino acids, vitamins, inorganic salts and glucose.

3. The method of claim 2, wherein the individualized cancer cell support media further comprises:
antibiotics and a buffering system suitable for maintaining a pH between 7.2 and 7.4.

4. The method of claim 1, further comprising the step of:
wherein the step of incubating the cancer cells in the individualized cancer cell support media takes place for a period of 30 minutes to four hours.

5. (canceled)

6. The method of claim 1, wherein the atmosphere contains 5% carbon dioxide and the step of incubating the cancer cells in the individualized cancer cell support media takes place at 37° C.

7. The method of claim 1, further comprising the steps of:
wherein the step of incubating the cancer cells in the individualized cancer cell support media at a temperature between 35° C. and 39° C. takes place under an atmosphere containing from 1% carbon dioxide to about 10% carbon dioxide for a period of 12 hours to 18 hours; and,
the step of treating the cancer cells with a compound of interest takes place for a period of about 30 minutes to four hours.

8. The method of claim 1, wherein the cancer cells support media further comprises patient derived serum and/or plasma.

9. (canceled)

10. (canceled)

11. (canceled)

12. A method for controlling the metabolic pathways used by cells during in vitro testing comprising:
identifying a cancer cell type to be treated;
identifying an organ from which the cancer cells were obtained, the organ having an in vivo environment which includes amino acids, vitamins and inorganic salts in various concentrations;
determining the amino acids, vitamins and inorganic salts and concentrations of the amino acids, vitamins and inorganic salts in the in vivo environment of the organ from which the cancer cells were obtained;
culturing cells of the identified cancer cell type in a patient specific cell support media formulated such that the patient specific support media corresponds to the in vivo environment of an organ within the patient that corresponds to the cancer cell type;
screening at least one compound of interest to ensure that the compound does not inhibit a luciferase enzyme;
upon confirmation that the at least one compound of interest does not inhibit the luciferase enzyme, treating the cells of the identified cancer cell type with the at least one compound of interest over a period of time that is less than two hours; and,
after treating the cells of the identified cancer cell type, monitoring the ATP level of the cells of the identified cancer cell type to determine cytotoxicity of the at least one compound of interest towards the cells of the identified cancer cell type.

13. The method of claim 12, wherein the step of treating the cells of the identified cancer cell type with the at least one compound of interest takes place over period of time that is less than three hours.

14. (canceled)

15. The method of claim 12, wherein the step of treating the cells of the identified cancer cell type with the at least one compound of interest does not result in cell death of the identified cancer cell type.

16. (canceled)

17. (canceled)

18. A method for assessing the in vitro effectiveness of a compound against a specific cancer type comprising:
obtaining cancer cells from an organ within a patient;
identifying an organ from which the cancer cells were obtained, the organ having an in vivo environment which includes amino acids, vitamins and inorganic salts in various concentrations;
determining the amino acids, vitamins and inorganic salts and concentrations of the amino acids, vitamins and inorganic salts in the in vivo environment of the organ from which the cancer cells were obtained;
preparing individualized cancer cell support media formulated to correspond to the in vivo environment within the organ within the patient from which the cancer cells were obtained;
incubating the cancer cells in the individualized cancer cell support media under conditions which will promote cancer cell growth using metabolic pathways corresponding to cancer cell growth in the patient wherein the cancer cells in the individualized cancer cell support media are maintained at a temperature between about 35° C. and 39° C. under an atmosphere containing carbon dioxide;
treating the cancer cells with at least one compound of interest for a period of about 30 minutes to four hours;
measuring a change in ATP levels of the cancer cells; and,
determining an IC₅₀ value for the compound of interest thereby assessing the in vitro effectiveness of a compound against a specific cancer type.

19. The method of claim 18, wherein the individualized cancer cell support media comprises:
amino acids, vitamins, inorganic salts and glucose.

20. The method of claim 19, wherein the individualized cancer cell support media further comprises:
antibiotics and a buffering system suitable for maintaining a pH between 7.2 and 7.4.

21. The method of claim 18, further comprising the step of:
wherein the step of incubating the cancer cells in the individualized cancer cell support media takes place for a period of 12 hours to 18 hours.

22. (canceled)

23. The method of claim 18, wherein the atmosphere contains 5% carbon dioxide and the step of incubating the cancer cells in the individualized cancer cell support media takes place at 37° C.

24. The method of claim 18, wherein the step of incubating the cancer cells in the individualized cancer cell support media between 35° C. and 39° C. takes place under an atmosphere containing from 1% carbon dioxide to about 10% carbon dioxide.

25. The method of claim 18, wherein the cancer cells support media further comprises patient derived serum and/or plasma.

26. (canceled)

27. (canceled)

28. A method for assessing the in vitro effectiveness of a compound against a specific cancer type comprising:
obtaining cancer cells from an organ within a patient;
identifying the organ from which the cancer cells were obtained, the organ having an in vivo environment;
preparing individualized cancer cell support media formulated to correspond to the in vivo environment within the organ within the patient from which the cancer cells were obtained;
maintaining the cancer cells in the individualized cancer cell support media at a temperature between about 35° C. and 39° C. under an atmosphere containing carbon dioxide;
treating the cancer cells with at least one compound of interest;
measuring a change in ATP levels of the cancer cells; and,
determining an IC₅₀ value for the compound of interest thereby assessing the in vitro effectiveness of a compound against a specific cancer type.

29. The method of claim 28, wherein the step of measuring a change in ATP levels of the cancer cells includes the steps of:
adding a luminescing agent to the cancer cells; and,
measuring a level of light emission of the luminescing agent and comparing the level of light emission to a level of light emission of a control.

30. The method of claim 28, wherein the step of measuring a change in ATP levels of the cancer cells includes the steps of:
adding a luminescing agent to the cancer cells;
measuring a level of fluorescence of the luminescing agent and comparing the level of fluorescence to a level of fluorescence of a control;

31. The method of claim 28, wherein the step of treating the cancer cells with at least one compound of interest occurs for a period of about 30 minutes to four hours.
